(19) United States
(12) Patent Application Publication
(10) Pub. No.: US 2022/0235082 A1
(43) Pub. Date: Jul. 28, 2022

(54) CATALYSTS

(71) Applicants: Goldenkeys High-Tech Materials Co., Ltd., Guian District, Guizhou (CN); The University of Warwick, Coventry, West Midlands (GB)

(72) Inventors: Martin Wills, Coventry, West Midlands (GB); Jonathan Barrios-Rivera, Coventry, West Midlands (GB); Yingjian Xu, Guian District, Guizhou (CN)

(21) Appl. No.: 17/614,183

(22) PCT Filed: May 27, 2020
(86) PCT No.: PCT/GB2020/051280

§ 371 Date: Nov. 24, 2021

(30) Foreign Application Priority Data
May 28, 2019 (GB) 1907506.8

(51) Int. Cl. C07F 15/00; B01J 31/18

(52) U.S. Cl. CPC C07F 15/0046; B01J 31/182

(57) ABSTRACT

A compound, e g a diamine ligand, represented by the following general formula (1): (Formula (1)) wherein each * represents an asymmetric carbon atom; X represents a group selected from one of an ester (e.g. a t-butyl ester); a thioester; an amide; a heterocyclic moiety (e.g. a five-membered heterocyclic ring) comprising one or more of O, S, Se, and/or P (e.g. a furan, a tetrahydrofuran, a thiophene, an isoxazole, a bromo-furan, or a thiazole); a moiety (e.g. a five-membered heterocyclic ring) comprising a nitrogen atom, wherein the nitrogen atom is protected with a protecting group containing an electron-withdrawing group, preferably the protecting group is selected from one of a carbamate protecting group, an amide protecting group, an aryl sulphonamide protecting group, or an alkyl sulphonamide protecting group; and optionally X may additionally comprise a solid support, e.g. a polymeric or a silica particle; Y represents or is CtT′T″ where ‘t’ is 0 or 1 and when ‘t’ is 1 T′ and T″ may individually represent a substituent, e.g. if t is 1, T′ and/or T″ may each be hydrogen or deuterium atom, or a halogen atom; for example, Y may represent a carbon atom comprising two further substituents; Z represents a hydrogen atom or a deuterium atom; R¹ represents an alkyl group (e.g. a functionalised alkyl group) preferably having between 1 to 100 carbon atoms, for example, between 1 to 30 carbon atoms (e.g. 1 to 20 carbon atoms, or 1 to 10 carbon atoms), a halogenated alkyl group preferably having between 1 to 100 carbon atoms (e.g. CF₃), for example, between 1 to 30 carbon atoms (e.g. 1 to 20 carbon atoms, or 1 to 10 carbon atoms), an aryl group preferably having between 5 to 100 carbon atoms, e.g. 6 to 30 carbon atoms and optionally having one or more substituents selected from alkyl groups preferably having 1 to 100 carbon atoms, e.g. 1 to 10 carbon atoms, halogenated alkyl groups preferably having 1 to 100 carbon atoms, e.g. 1 to 10 carbon atoms, and/or halogen atoms; or R¹ represents a solid support, e.g. a silica particle or a polymeric particle; R² and R³ each independently represent a group selected from alkyl groups preferably having between 1 to 100 carbon atoms, for example 1 to 20 carbon atoms (e.g. 1 to 10 carbon atoms), aryl groups (e.g. phenyl groups), and cycloalkyl groups preferably having 3 to 8 carbon atoms, the aryl group or phenyl group optionally having one or more substituents selected from alkyl groups preferably having between 1 to 100 carbon atoms, e.g. between 1 to 20 carbon atoms (e.g. 1 to 10 carbon atoms), alkoxy groups preferably having between 1 to 100 carbon atoms, for example, between 1 to 20 carbon atoms (e.g. 1 to 10 carbon atoms), and halogen atoms, and each hydrogen atom of the cycloalkyl groups being optionally replaced by an alkyl group preferably having between 1 to 100 carbon atoms, e.g. 1 to 20 carbon atoms (e.g. 1 to 10 carbon atoms), or R¹ represents a polyethylene glycol (PEG) moiety having the formula C₂ₙH₄ₙ₊₂Oₙ₊₁ wherein n is an integer between 1 and 100; or R² and R³ form a ring together with carbon atoms to which R² and R³ are bonded; R⁴ represents a hydrogen atom or a deuterium atom.

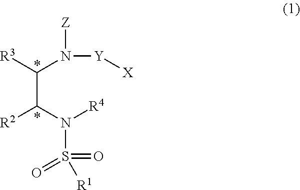

This invention relates generally to organometallic catalysts comprising diamine ligands. More specifically, although not exclusively, this invention relates to tosylated-1,2-diphenyl-ethane-1,2-diamine (TsDPEN) ligands, metal complexes containing tosylated-1,2-diphenyl-ethane-1,2-diamine ligands (TsDPEN), and the use of said metal complexes as organometallic catalysts, for example, for use in asymmetric transfer hydrogenation reactions.

Asymmetric catalysis, i.e. catalysis in which a chiral catalyst directs the formation of a chiral compound such that formation of one particular stereoisomer is favoured, is an active area of research that finds particular application for the synthesis of pharmaceuticals and their precursors.

N-(p-Toluenesulphonyl)-1,2-diphenylethylene-1,2-diamine (TsDPEN) is a widely used chiral diamine derivative with synthetic applications in asymmetric catalysis. One of the most successful and established catalysis applications of TsDPEN is as a ligand in [η⁶-arene)Ru(II)TsDPEN(Cl)] complexes, which are now established as efficient and selective catalysts for asymmetric transfer hydrogenation (ATH) of ketones and imines (for example, see R. Noyori and S. Hashiguchi, Acc. Chem. Res., 1997, 30, 97-102).

In the majority of reported examples of [η⁶-arene)Ru(II)TsDPEN(Cl)] catalysts, the TsDPEN ligand contains a primary amine. It is known to functionalise the primary amine, for example by alkylation, without significantly degrading the reactivity or selectivity of the corresponding catalyst (for example, see Wills et. al. Tetrahedron: Asymmetry 2010, 21, 2258-2264 and Wills et. al. Org. Lett., 2009, 11 (4), pp 847-850).

It is also known to functionalise the primary amine of TsDPEN with an appropriate moiety to form a tridentate structure in a metal complex. Typically, the moiety is a triazole or a pyridine group, which acts as a ‘third donor group’ to donate electron density into the metal centre in addition to the two secondary amines of the ligand. However, it has been found in the course of an unpublished research project that [η⁶-arene)Ru(II)TsDPEN(Cl)] catalysts comprising this type of ligand are poor catalysts because the third donor group is likely to strongly coordinate to the metal centre, which inhibits the catalytic activity.

Despite this issue, such ligands have been demonstrated to form active catalysts when used with Ru₃(CO)₁₂ to form complexes in situ (for example, see Wills et. al. Org. Lett., 2012, 14 (20), pp 5230-5233)

Although many ligands and corresponding metal complexes have been proposed for use in asymmetric catalysis, it remains a challenge to provide catalysts for the asymmetric reduction of substrates with a wide range of structures and functionality, in high enantioselectivity (ee) and yield. For example, it is a particular challenge to provide catalysts for use in asymmetric transfer hydrogenation of ortho-substituted phenyl ketones (see Wills et. al. Org. Lett., 2012, 14 (20), pp 5230-5233). Therefore, it would be advantageous to provide asymmetric catalysts that are able to reduce more complex substrates in high ee values.

It would also be advantageous to provide tridentate TsDPEN ligands with a third donor group, for use in conventional systems, that is, for use in [η⁶-arene)Ru(II)TsDPEN(Cl)] complexes. Although the use of Ru₃(CO)₁₂ with such ligands has been shown to be effective at reducing substrates in moderate enantioselectivity, it would be advantageous to provide a single reagent for use in asymmetric transfer hydrogenations, which is able to reduce substrates with more complex structures and/or functionality in high enantiomeric excess.

It is therefore a first non-exclusive object of the invention to provide a TsDPEN ligand for use in an organometallic asymmetric catalyst, wherein the catalyst is for use in the asymmetric reduction of substrates comprising complex functionality.

Accordingly, a first aspect of the invention provides a compound, e.g. a diamine ligand, represented by the following general formula (1):

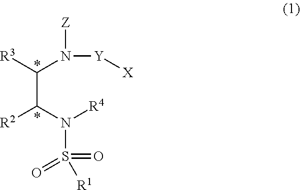

wherein each * represents an asymmetric carbon atom;

X represents a group selected from one of an ester (e.g. a t-butyl ester); a thioester; an amide; a heterocyclic moiety (e.g. a five-membered heterocyclic ring) comprising one or more of O, S, Se, and/or P (e.g. a furan, a tetrahydrofuran, a thiophene, an isoxazole, a bromo-furan, or a thiazole); a moiety (e.g. a five-membered heterocyclic ring) comprising a nitrogen atom, wherein the nitrogen atom is protected with a protecting group containing an electron-withdrawing group, preferably the protecting group is selected from one of a carbamate protecting group, an amide protecting group, an aryl sulphonamide protecting group, or an alkyl sulphonamide protecting group; and optionally X may additionally comprise a solid support, e.g. a polymeric or a silica particle;

Y represents or is CₜT′T″ where ‘t’ is 0 or 1 and when T is 1 T′ and T″ may individually represent a substituent, e.g. if t is 1, T′ and/or T″ may each be hydrogen or deuterium atom, or a halogen atom, for example Y may represent CH₂ and, for example if t is 0 then Y is not present;

Z represents a hydrogen atom or a deuterium atom;

R¹ represents an alkyl group (e.g. a functionalised alkyl group) preferably having between 1 to 100 carbon atoms, for example, between 1 to 30 carbon atoms (e.g. 1 to 20 carbon atoms, or 1 to 10 carbon atoms), a halogenated alkyl group preferably having between 1 to 100 carbon atoms (e.g. CF₃), for example, between 1 to 30 carbon atoms (e.g. 1 to 20 carbon atoms, or 1 to 10 carbon atoms), an aryl group preferably having between 5 to 100 carbon atoms, e.g. 6 to 30 carbon atoms and optionally having one or more substituents selected from alkyl groups preferably having 1 to 100 carbon atoms, e.g. 1 to 10 carbon atoms, halogenated alkyl groups preferably having 1 to 100 carbon atoms, e.g. 1 to 10 carbon atoms, and/or halogen atoms; or R¹ represents a polyethylene glycol (PEG) moiety having the formula C₂ₙH₄ₙ₊₂Oₙ₊₁ wherein n is an integer between 1 and 100; or R¹ represents a solid support, e.g. a silica particle or a polymeric particle;

R² and R³ each independently represent a group selected from alkyl groups preferably having between 1 to 100 carbon atoms, for example 1 to 20 carbon atoms (e.g. 1 to 10 carbon atoms), aryl groups (e.g. phenyl groups), and cycloalkyl groups preferably having 3 to 8 carbon atoms, the aryl group or phenyl group optionally having one or more substituents selected from alkyl groups preferably having between 1 to 100 carbon atoms, e.g. between 1 to 20 carbon atoms (e.g. 1 to 10 carbon atoms), alkoxy groups preferably having between 1 to 100 carbon atoms, for example, between 1 to 20 carbon atoms (e.g. 1 to 10 carbon atoms), and halogen atoms, and each hydrogen atom of the cycloalkyl groups being optionally replaced by an alkyl group preferably having between 1 to 100 carbon atoms, e.g. 1 to 20 carbon atoms (e.g. 1 to 10 carbon atoms), or R² and R³ form a ring together with carbon atoms to which R² and R³ are bonded;

R⁴ represents a hydrogen atom or a deuterium atom.

A further aspect of the invention provides a compound, e.g. a diamine ligand, represented by the following general formula (1):

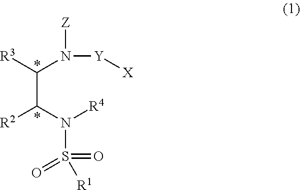

wherein
each * represents an asymmetric carbon atom;
X represents one of C═O, C—S, C—O, or a heterocyclic moiety comprising one or more of O, S, Se, and/or P;
Y represents a carbon atom comprising two further substituents, e.g. Y represents CH₂;
Z represents a hydrogen atom or a deuterium atom;
R¹ represents an alkyl group (e.g. a functionalised alkyl group) having between 1 to 20 carbon atoms (e.g. 1 to 10 carbon atoms), halogenated alkyl groups having 1 to 20 carbon atoms (e.g. 1 to 10 carbon atoms), an aryl group having 6 to 30 carbon atoms and optionally having one or more substituents selected from alkyl groups having 1 to 10 carbon atoms, halogenated alkyl groups having 1 to 10 carbon atoms, and/or halogen atoms;
R² and R³ each independently represent a group selected from alkyl groups having 1 to 20 carbon atoms (e.g. 1 to 10 carbon atoms), aryl groups (e.g. phenyl groups), and cycloalkyl groups having 3 to 8 carbon atoms, the phenyl groups optionally having one or more substituents selected from alkyl groups having 1 to 20 carbon atoms (e.g. 1 to 10 carbon atoms), alkoxy groups having 1 to 20 carbon atoms (e.g. 1 to 10 carbon atoms), and halogen atoms, and each hydrogen atom of the cycloalkyl groups being optionally replaced by an alkyl group having 1 to 20 carbon atoms (e.g. 1 to 10 carbon atoms), or R² and R³ form a ring together with carbon atoms to which R² and R³ are bonded;
R⁴ represents a hydrogen atom or a deuterium atom.

It has been surprisingly found that compounds according to the general formula (1) are usable as diamine ligands to form metal complexes for use as catalysts in asymmetric transfer hydrogenations. Without wishing to be bound by any particular theory, the inventors have found that compounds wherein X is defined as above are “weak donors”, which comprise atoms that are unable to form tridentate ligands in (areneRu) complexes. These are catalytically active, in comparison to those ligands with “strong” donors (e.g. wherein X comprises a pyridine group or a triazole group), which form inactive catalysts. This is surprising because the inventors initially predicted that compounds according to the general formula (1) would also form inactive tridentate catalysts. However, ligands and complexes according to the invention have been shown to be usable in asymmetric hydrogenation reactions to reduce substrates in high yield and enantioselectivity.

In embodiments, X represents a heterocyclic moiety comprising one or more of O, S, Se, and/or P. In embodiments, X represents a five-membered heterocyclic group or moiety comprising one or more of O, S, Se, and/or P, e.g. a furan, a tetrahydrofuran, a thiophene, an isoxazole, a bromo-furan, or a thiazole.

In embodiments, X represents an aromatic five-membered heterocyclic group or moiety comprising one or more of O, S, Se, and/or P. In embodiments, X represents a non-aromatic five-membered heterocyclic group or moiety comprising one or more of O, S, Se, and/or P (that is, a moiety that does not obey Hackers Rule).

In embodiments, X represents a heterocyclic moiety comprising one or more of O, S, Se, and/or P, wherein the heterocyclic moiety comprises one or more additional substituents (for example, one, two, three, or four, or more additional substituents that are the same or different from one another). For example, X may represent a five-membered heterocyclic moiety comprising one or more additional substituents, e.g. a further substituted furan, a thiophene, an isoxazole, a bromo-furan, or a thiazole. In embodiments, the substituted heterocyclic moiety may be mono-substituted with a further aliphatic or aromatic moiety. In embodiments, the substituted heterocyclic moiety may be di-substituted, tri-substituted, tetrasubstituted with further substituents that are the same or different, each of which may be an aliphatic or aromatic moiety.

In embodiments, X does not represent a thiazole.

In embodiments, X does not represent a Brønsted acid. In embodiments, X does not comprise and/or represent a COOH moiety.

In embodiments, X represents a moiety containing one or more nitrogen atoms. In embodiments, X does not represent a moiety containing a nitrogen atom.

In embodiments, X represents an ester, e.g. a t-butyl ester. By X represents an ester, we mean that X represents a moiety comprising —(C═O)—O—R^{x} wherein R^{x} is an alkyl moiety comprising between 1 to 10 carbon atoms, for example, between 1, 2, 3, 4, 5, 6, 7, 8, 9 carbon atoms to 10, 9, 8, 7, 6, 5, 4, 3, 2 carbon atoms, e.g. R^{x} is a t-butyl group.

In embodiments, X represents a thioester, e.g. —(C═O)—SR^{x}, wherein R^{x} is an alkyl moiety comprising between 1 to 10 carbon atoms, for example, between 1, 2, 3, 4, 5, 6, 7, 8, 9 carbon atoms to 10, 9, 8, 7, 6, 5, 4, 3, 2 carbon atoms, e.g. R^{x} is a t-butyl group.

In embodiments, X represents an amide, e.g. —(C═O)—NR^{x}R^{y} or —N(R^{x})—(C═O)R^{y}, wherein R^{x} and/or R^{y} independently represent an alkyl moiety comprising between 1 to 10 carbon atoms, for example, between 1, 2, 3, 4, 5, 6, 7, 8, 9 carbon atoms to 10, 9, 8, 7, 6, 5, 4, 3, 2 carbon atoms, e.g. R^{x} is a t-butyl group.

In embodiments, X represents a moiety (e.g. a five-membered heterocyclic ring) comprising a nitrogen atom, wherein the nitrogen atom is protected with a protecting group selected from one of a carbamate protecting group, an amide protecting group, an aryl sulphonamide protecting group, or an alkyl sulphonamide protecting group.

In embodiments, X represents an alkyl moiety comprising a nitrogen atom, wherein the nitrogen atom is protected with a protecting group selected from one of a carbamate protecting group, an amide protecting group, an aryl sulphonamide protecting group, or an alkyl sulphonamide protecting group. That is, X represents an alkyl moiety comprising between 1 to 10 carbon atoms, for example, between any one of 1, 2, 3, 4, 5, 6, 7, 8, or 9 to any one of 10, 9, 8, 7, 6, 5, 4, 3, or 2 carbon atoms, e.g. 1, 2, 3, 4, 5, 6, 7, 8, 9, or 10 carbon atoms. The alkyl moiety may be linear, branched, or cyclic. The alkyl moiety may comprise one or more substituents, e.g. alkyl substituents comprising between 1 to 10 carbon atoms; or halide substituents.

In embodiments, X represents an aryl moiety comprising a nitrogen atom, wherein the nitrogen atom is protected with a protecting group selected from one of a carbamate protecting group, an amide protecting group, an aryl sulphonamide protecting group, or an alkyl sulphonamide protecting group. That is, X represents an aryl moiety comprising between 4 to 18 carbon atoms, for example, between any one of 4, 5, 6, 7, 8, 9, 10, 11, 12, 13, 14, 15, 16, or 17 to any one of 18, 17, 16, 15, 14, 13, 12, 11, 10, 9, 8, 7, 6 or 5 carbon atoms, e.g. 4, 5, 6, 7, 8, 9, 10, 11, 12, 13, 14, 15, 16, 17, or 18 carbon atoms. The aryl moiety may comprise one or more substituents, e.g. alkyl substituents comprising between 1 to 10 carbon atoms; and/or halide substituents.

In embodiments, X represents an alkyl heterocyclic moiety (e.g. a five-membered heterocyclic ring, for example a five membered heterocyclic ring) comprising a nitrogen atom, wherein the nitrogen atom is protected with a protecting group selected from one of a carbamate protecting group, an amide protecting group, an aryl sulphonamide protecting group, or an alkyl sulphonamide protecting group.

In embodiments, X represents a pyrrolidine moiety comprising a tert-butoxycarbonyl protecting group (BOC group).

In embodiments, X represents a pyrrolidine moiety comprising an amide protecting group N—(C═O)R^{x}, wherein R^{x} represents an alkyl moiety comprising between 1 to 20 carbon atoms, for example, between 1, 2, 3, 4, 5, 6, 7, 8, 9, 10, 11, 12, 13, 14, 15, 16, 17, 18, 19 carbon atoms to 20, 19, 18, 17, 16, 15, 14, 13, 12, 11, 10, 9, 8, 7, 6, 5, 4, 3, 2 carbon atoms.

In embodiments, X represents a pyrrolidine moiety comprising an aryl sulphonamide protecting group N—S(O)₂R^{y} wherein R^{y} represents an aryl moiety comprising between 5 to 18 carbon atoms, for example, between any one of 5, 6, 7, 8, 9, 10, 11, 12, 13, 14, 15, 16, or 17 to any one of 18, 17, 16, 15, 14, 13, 12, 11, 10, 9, 8, 7, or 6 carbon atoms, e.g. 5, 6, 7, 8, 9, 10, 11, 12, 13, 14, 15, 16, 17, or 18 carbon atoms. The aryl moiety may comprise one or more substituents, e.g. alkyl substituents comprising between 1 to 10 carbon atoms; and/or halide substituents.

In embodiments, X represents a pyrrolidine moiety comprising an alkyl sulphonamide protecting group N—S(O)₂R^{z} wherein R^{z} represents an alkyl moiety comprising between 1 to 20 carbon atoms, for example, between 1, 2, 3, 4, 5, 6, 7, 8, 9, 10, 11, 12, 13, 14, 15, 16, 17, 18, 19 carbon atoms to 20, 19, 18, 17, 16, 15, 14, 13, 12, 11, 10, 9, 8, 7, 6, 5, 4, 3, 2 carbon atoms.

In embodiments, Y is not present meaning that the compound has the following general formula:

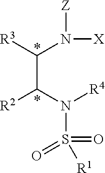

In embodiments, Y is a carbon atom comprising two hydrogen substituents, i.e. CH₂. In alternative embodiments, Y is a carbon atom comprising two substituents, one or both may be a hydrogen atom, a deuterium atom, a halogen atom (e.g. fluorine, chlorine, bromine, iodine). In embodiments, Y represent a carbon atom further comprising an alkyl group and/or an aryl group.

In embodiments, Y represents a CH₂ moiety and X represents a group selected from an ester (e.g. a t-butyl ester); a thioester; an amide; a heterocyclic moiety (e.g. a five-membered heterocyclic ring) comprising one or more of 0, S, Se, and/or P (e.g. a furan, a tetrahydrofuran, a thiophene, an isoxazole, a bromo-furan, or a thiazole).

In embodiments, R¹ represents alkyl group (e.g. a functionalised alkyl group) or a halogenated alkyl group, wherein the alkyl group or the halogenated alkyl group have between 1 to 100 carbon atoms (e.g. CF₃), for example, between 1 to 90, between 1 to 80 carbon atoms, between 1 to 70 carbon atoms, between 1 to 60 carbon atoms, between 1 to 50 carbon atoms, between 1 to 40 carbon atoms, between 1 to 30 carbon atoms, between 1 to 20 carbon atoms, or 1 to 10 carbon atoms.

In embodiments, R¹ represents an aryl group having between 5 to 100 carbon atoms, for example, between 5 to 90 carbon atoms, between 5 to 80 carbon atoms, between 5 to 70 carbon atoms, between 5 to 60 carbon atoms, between 5 to 50 carbon atoms, between 5 to 40 carbon atoms, or between 5 to 30 carbon atoms, e.g. between 6 to 30 carbon atoms. Optionally, wherein R¹ represents an aryl group, the aryl group has one or more substituents selected from alkyl groups having between 1 to 100 carbon atoms, for example, between 1 to 90 carbon atoms, between 1 to 80 carbon atoms, between 1 to 70 carbon atoms, between 1 to 60 carbon atoms, between 1 to 50 carbon atoms, between 1 to 40 carbon atoms, between 1 to 30 carbon atoms, between 1 to 20 carbon atoms, or 1 to 10 carbon atoms; or halogenated alkyl groups having between 1 to 100 carbon atoms, for example, between 1 to 90 carbon atoms, between 1 to 80 carbon atoms, between 1 to 70 carbon atoms, between 1 to 60 carbon atoms, between 1 to 50 carbon atoms, between 1 to 40 carbon atoms, between 1 to 30 carbon atoms, between 1 to 20 carbon atoms, or 1 to 10 carbon atoms.

In embodiments, R² and R³ each independently represent a group selected from alkyl groups preferably having between 1 to 100 carbon atoms, for example between 1 to 90 carbon atoms, between 1 to 80 carbon atoms, between 1 to 70 carbon atoms, between 1 to 60 carbon atoms, between 1 to 50 carbon atoms, between 1 to 40 carbon atoms, between 1 to 30 carbon atoms, between 1 to 20 carbon atoms, or 1 to 10 carbon atoms.

In embodiments wherein R² and R³ each independently represent an aryl group or a phenyl group, the aryl group or phenyl group optionally has one or more substituents selected from alkyl groups preferably having between 1 to 100 carbon atoms, between 1 to 90 carbon atoms, between 1 to 80 carbon atoms, between 1 to 70 carbon atoms, between 1 to 60 carbon atoms, between 1 to 50 carbon atoms, between 1 to 40 carbon atoms, between 1 to 30 carbon atoms, between 1 to 20 carbon atoms, or 1 to 10 carbon atoms.

In embodiments wherein R² and R³ each independently represent an alkoxy group, the alkoxy groups preferably has between 1 to 100 carbon atoms, for example, between 1 to 90 carbon atoms, between 1 to 80 carbon atoms, between 1 to 70 carbon atoms, between 1 to 60 carbon atoms, between 1 to 50 carbon atoms, between 1 to 40 carbon atoms, between 1 to 30 carbon atoms, between 1 to 20 carbon atoms, or 1 to 10 carbon atoms.

In embodiments wherein each hydrogen atom of the cycloalkyl groups is optionally replaced by an alkyl group, the alkyl group preferably has between 1 to 100 carbon atoms, between 1 to 90 carbon atoms, between 1 to 80 carbon atoms, between 1 to 70 carbon atoms, between 1 to 60 carbon atoms, between 1 to 50 carbon atoms, between 1 to 40 carbon atoms, between 1 to 30 carbon atoms, between 1 to 20 carbon atoms, or 1 to 10 carbon atoms.

In embodiments, R¹ represents a polyethylene glycol (PEG) moiety having the formula C₂ₙH₄ₙ₊₂Oₙ₊₁ wherein n is an integer between 1 and 100, e.g. between 1 to 90, 1 to 80, 1 to 70, 1 to 60, 1 to 50, 1 to 40, 1 to 30, 1 to 20, 1 to 10, e.g. 1, 2, 3, 4, 5, 6, 7, 8, 9, 0110.

In embodiments, R¹ represents a C₆H₄CH₃ moiety. In embodiments, R¹ represents a CF₃ moiety. In embodiments, R¹ represents a polymer particle or a silica particle. In embodiments, R² and R³ each represent a phenyl moiety.

Accordingly, a further aspect of the invention provides a compound, e.g. a diamine ligand, represented by the following general formula (1A, 1B, 1C, 1D):

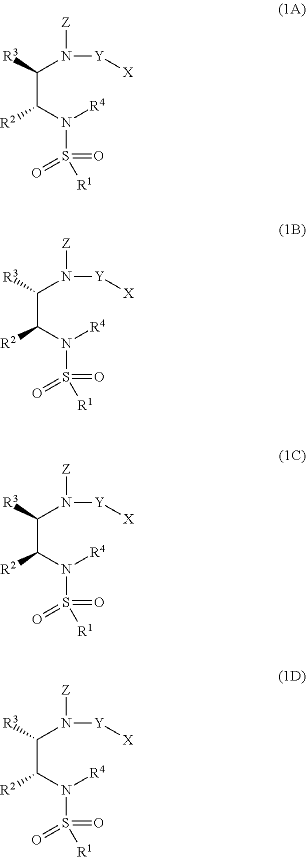

wherein X, Y, Z, R¹, R², R³, and R⁴ are defined as above.

In embodiments, R¹ represents 4-methylbenzene or CH₃ or CF₃ or 2,4,6-triisopropylbenzene. In embodiments, R¹ represents a solid support, e.g. a silica particle or a polymeric particle.

In embodiments, R² and R³ each represent a substituted or non-substituted phenyl group in the general formula (1E):

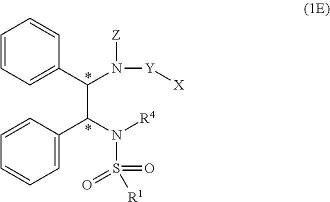

wherein each * represents an asymmetric carbon atom; and

X, Y, Z, R¹, and R⁴ are defined as above.

In embodiments, the asymmetric carbon atoms are represented by the general formula (1F), (1G), (1H), or (1J):

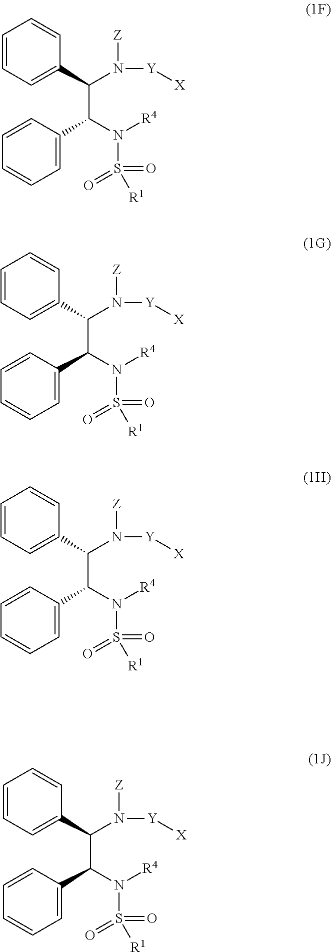

wherein X, Y, Z, R¹, and R⁴ are defined as above.

A further aspect of the invention provides a metal-diamine complex, for example a transition metal-diamine complex represented by the general formula (2).

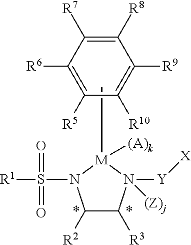

wherein

M represents a transition metal catalyst, and/or one of ruthenium, iridium, rhodium, or osmium;

each * represents an asymmetric carbon atom;

X represents a group selected from one of an ester (e.g. a t-butyl ester); a thioester; an amide; a heterocyclic moiety (e.g. a five-membered heterocyclic ring) comprising one or more of O, S, Se, and/or P (e.g. a furan, a tetrahydrofuran, a thiophene, an isoxazole, a bromo-furan, or a thiazole); a moiety (e.g. a five-membered heterocyclic ring) comprising a nitrogen atom, wherein the nitrogen atom is protected with a protecting group containing an electron-withdrawing group, preferably the protecting group is selected from one of a carbamate protecting group, an amide protecting group, an aryl sulphonamide protecting group, or an alkyl sulphonamide protecting group; and optionally X may additionally comprise a solid support, e.g. a polymeric or a silica particle;

Y represents or is CₜT′T″ where ‘t’ is 0 or 1 and when T is 1 T′ and T″ may individually represent a substituent, e.g. if t is 1, T′ and/or T″ may each be hydrogen or deuterium atom, or a halogen atom, for example Y may represent CH₂;

Z represents a hydrogen atom or a deuterium atom;

R¹ represents an alkyl group (e.g. a functionalised alkyl group) preferably having between 1 to 100 carbon atoms, for example, between 1 to 30 carbon atoms (e.g. 1 to 20 carbon atoms, or 1 to 10 carbon atoms), a halogenated alkyl group preferably having between 1 to 100 carbon atoms (e.g. CF₃), for example, between 1 to 30 carbon atoms (e.g. 1 to 20 carbon atoms, or 1 to 10 carbon atoms), an aryl group preferably having between 5 to 100 carbon atoms, e.g. 6 to 30 carbon atoms and optionally having one or more substituents selected from alkyl groups preferably having 1 to 100 carbon atoms, e.g. 1 to 10 carbon atoms, halogenated alkyl groups preferably having 1 to 100 carbon atoms, e.g. 1 to 10 carbon atoms, and/or halogen atoms; or R¹ represents a polyethylene glycol (PEG) moiety having the formula C₂ₙH₄ₙ₊₂Oₙ₊₁ wherein n is an integer between 1 and 100; or R¹ represents a solid support, e.g. a silica particle or a polymeric particle;

R² and R³ each independently represent a group selected from alkyl groups preferably having between 1 to 100 carbon atoms, for example 1 to 20 carbon atoms (e.g. 1 to 10 carbon atoms), aryl groups (e.g. phenyl groups), and cycloalkyl groups preferably having 3 to 8 carbon atoms, the aryl group or phenyl group optionally having one or more substituents selected from alkyl groups preferably having between 1 to 100 carbon atoms, e.g. between 1 to 20 carbon atoms (e.g. 1 to 10 carbon atoms), alkoxy groups preferably having between 1 to 100 carbon atoms, for example, between 1 to 20 carbon atoms (e.g. 1 to 10 carbon atoms), and halogen atoms, and each hydrogen atom of the cycloalkyl groups being optionally replaced by an alkyl group preferably having between 1 to 100 carbon atoms, e.g. 1 to 20 carbon atoms (e.g. 1 to 10 carbon atoms), or R² and R³ form a ring together with carbon atoms to which R² and R³ are bonded;

R⁵, R⁶, R⁷, R⁸, R⁹, and R¹⁰ each independently represent a group selected from a hydrogen atom, a deuterium atom, alkyl groups preferably having 1 to 100 carbon atoms, e.g. 1 to 10 carbon atoms, silyl groups having 1 to 3 alkyl groups preferably having 1 to 100 carbon atoms, e.g. 10 carbon atoms, alkoxy groups preferably having 1 to 100 carbon atoms, e.g. 1 to 10 carbon atoms, and C(═O)—OR¹¹, where R¹¹ represents an alkyl group preferably having 1 to 100 carbon atoms, e.g. 1 to 10 carbon atoms, a heteroaryl group preferably having 4 to 100 carbon atoms, e.g. 4 to 10 carbon atoms, or an aryl group preferably having 6 to 100 carbon atoms, e.g. 6 to 10 carbon atoms;

A represents a group selected from a trifluoromethanesulfonyloxy group, a p-toluenesulfonyloxy group, a methanesulfonyloxy group, a benzenesulfonyloxy group, a hydrogen atom, a halogen atom, acetate, hexafluorophosphate, and tetrafluoroborate;

j and k each represent 0 or 1, but j+k is not 1.

In embodiments, one or more of R⁵, R⁶, R⁷, R⁸, R⁹, and/or R¹⁰ may each independently represent an alkyl groups having between 1 to 100 carbon atoms, e.g. between 1 to 90 carbon atoms, between 1 to 80 carbon atoms, between 1 to 70 carbon atoms, between 1 to 60 carbon atoms, between 1 to 50 carbon atoms, between 1 to 40 carbon atoms, between 1 to 30 carbon atoms, between 1 to 20 carbon atoms, or between 1 to 10 carbon atoms.

In embodiments, one or more of R⁵, R⁶, R⁷, R⁸, R⁹, and/or R¹⁰ may each independently represent a silyl groups having 1 to 3 alkyl groups having 1 to 100 carbon atoms, e.g. between 1 to 90 carbon atoms, between 1 to 80 carbon atoms, between 1 to 70 carbon atoms, between 1 to 60 carbon atoms, between 1 to 50 carbon atoms, between 1 to 40 carbon atoms, between 1 to 30 carbon atoms, between 1 to 20 carbon atoms, or between 1 to 10 carbon atoms.

In embodiments, one or more of R⁵, R⁶, R⁷, R⁸, R⁹, and/or R¹⁰ may each independently represent a group of C(═O)—OR¹¹, where R¹¹ represents an alkyl group having 1 to 100 carbon atoms, between 1 to 90 carbon atoms, between 1 to 80 carbon atoms, between 1 to 70 carbon atoms, between 1 to 60 carbon atoms, between 1 to 50 carbon atoms, between 1 to 40 carbon atoms, between 1 to 30 carbon atoms, between 1 to 20 carbon atoms, or between 1 to 10 carbon atoms.

In embodiments, one or more of R⁵, R⁶, R⁷, R⁸, R⁹, and/or R¹⁰ may each independently represent a heteroaryl group having 4 to 100 carbon atoms, e.g. 4 to 90, 4 to 80, 4 to 70, 4 to 60, 4 to 50, 4 to 40, 4 to 30, 4 to 20, or 4 to 10 carbon atoms; or an aryl group having 6 to 100 carbon atoms, e.g. 6 to 90, 6 to 80, 6 to 70, 6 to 60, 6 to 50, 6 to 40, 4 to 30, 6 to 20, or 6 to 10 carbon atoms.

In embodiments, R¹ represents an alkyl group having between 1 to 10 carbon atoms, halogenated alkyl groups having 1 to 10 carbon atoms, an aryl group having 6 to 30 carbon atoms and optionally having one or more substituents selected from alkyl groups having 1 to 10 carbon atoms, halogenated alkyl groups having 1 to 10 carbon atoms, and/or halogen atoms; or R¹ represents a solid support, e.g. a silica particle or a polymeric particle;

R² and R³ each independently represent a group selected from alkyl groups having 1 to 10 carbon atoms, phenyl groups, and cycloalkyl groups having 3 to 8 carbon atoms, the phenyl groups optionally having one or more substituents selected from alkyl groups having 1 to 10 carbon atoms, alkoxy groups having 1 to 10 carbon atoms, and halogen atoms, and each hydrogen atom of the cycloalkyl groups being optionally replaced by an alkyl group having 1 to 10 carbon atoms, or R² and R³ form a ring together with carbon atoms to which R² and R³ are bonded;

R⁵, R⁶, R⁷, R⁸, R⁹, and R¹⁰ each independently represent a group selected from a hydrogen atom, a deuterium atom, alkyl groups having 1 to 10 carbon atoms, silyl groups having 1 to 3 alkyl groups having 1 to 10 carbon atoms, alkoxy groups having 1 to 10 carbon atoms, and C(═O)—OR¹¹, where R¹¹ represents an alkyl group having 1 to 10 carbon atoms, a heteroaryl group having 4 to 10 carbon atoms, or an aryl group having 6 to 10 carbon atoms;

A represents a group selected from a trifluoromethanesulfonyloxy group, a p-toluenesulfonyloxy group, a methanesulfonyloxy group, a benzenesulfonyloxy group, a hydrogen atom, a halogen atom, acetate, hexafluorophosphate, and tetrafluoroborate;

j and k each represent 0 or 1, but j+k is not 1.

A further aspect of the invention provides a metal-diamine complex, for example a transition metal-diamine complex represented by the general formula (3):

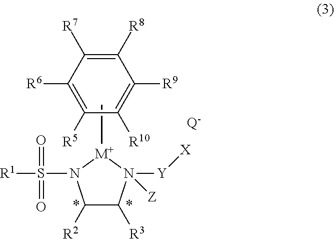

wherein each * represents an asymmetric carbon atom;

M, X, Y, Z, R¹, R², R³, R⁵, R⁶, R⁷, R⁸, R⁹, R¹⁰ are defined as above; and

Q represents a counter anion.

A further aspect of the invention provides a metal-diamine complex, for example a transition metal-diamine complex represented by the general formula (4):

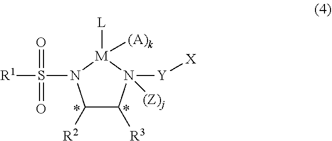

wherein each * represents an asymmetric carbon atom;

M, X, Y, Z, j, k, R¹, R², R³ are defined as above; and

L represents a cyclopentadienyl or pentamethylcyclopentadienyl ligand.

A further aspect of the invention provides a metal-diamine complex, for example a transition metal-diamine complex represented by the general formula (5):

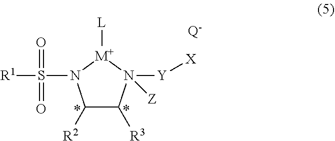

wherein each * represents an asymmetric carbon atom;

M, X, Y, Z, R¹, R², R³ are defined as above; and

Q represents a counter anion;

L represents a cyclopentadienyl or pentamethylcyclopentadienyl ligand.

In embodiments, R¹ in any of the above identified general formulae represents an alkyl group. The alkyl group having 1 to 10 carbon atoms represented by R¹ is preferably a linear or branched alkyl group having 1 to 5 carbon atoms. Specific examples of the alkyl group having 1 to 10 carbon atoms include a methyl group, an ethyl group, a n-propyl group, an isopropyl group, a n-butyl group, an isobutyl group, a s-butyl group, a t-butyl group, a n-pentyl group, a n-hexyl group, a n-heptyl group, a n-octyl group, a n-nonyl group, a n-decyl group, and the like. The alkyl group is preferably a methyl group, an ethyl group, a n-propyl group, an isopropyl group, a n-butyl group, an isobutyl group, a s-butyl group, a t-butyl group, or a n-pentyl group.

In embodiments, R¹ in any of the above identified general formulae represents a halogenated alkyl group. The halogenated alkyl group having 1 to 10 carbon atoms represented by R¹ is the same group as the above-described alkyl group having 1 to 10 carbon atoms, except that one or multiple hydrogen atoms are replaced by one or multiple halogen atoms. The halogenated alkyl group having 1 to 10 carbon atoms is preferably a linear or branched halogenated alkyl group having 1 to 5 carbon atoms. Examples of the halogen atoms include chlorine atoms, bromine atoms, fluorine atoms, and iodine atoms. Specific examples of the halogenated alkyl groups having 1 to 10 carbon atoms include a trifluoromethane group, a trichloromethane group, a tribromomethane group, and the like.

In embodiments, R¹ in any of the above identified general formulae represents an aryl group. The aryl group having 6 to 30 carbon atoms may be an aromatic monocyclic group, aromatic polycyclic group, or aromatic fused cyclic group having 6 to 30 carbon atoms, and is preferably an aromatic monocyclic group, aromatic polycyclic group, or aromatic fused cyclic group having 6 to 15 carbon atoms, and particularly preferably aromatic monocyclic group having 6 to 12 carbon atoms. Specific examples of the aryl group having 6 to 30 carbon atoms include a phenyl group, a naphthyl group, an anthryl group, a phenanthryl group, an indenyl group, and the like. A phenyl group is the most preferable. In addition, the aryl group optionally has one or more substituents selected from alkyl groups having 1 to 10 carbon atoms, halogenated alkyl groups having 1 to 10 carbon atoms, and halogen atoms.

The alkyl groups and the halogenated alkyl groups as the substituents can be selected from the above-described groups defined as the alkyl groups or the halogenated alkyl groups represented by R¹ in any of the above identified general formulae. Of these groups, linear alkyl groups having 1 to 5 carbon atoms are particularly preferable.

Examples of the halogen atoms include chlorine atoms, bromine atoms, fluorine atoms, and iodine atoms.

Specific examples of the aryl group represented by R¹ in any of the above identified general formulae and substituted with the substituents include a p-tolyl group, a 2,4,6-trimethylphenyl group, a 4-trifluoromethylphenyl group, a pentafluorophenyl group, and the like.

A yet further aspect of the invention provides a method of producing a metal complex represented by one of the general formulae (2), (3), (4), or (5), the method comprising reacting the ligand represented by the general formula (1), e.g. (1A) or (16), with a metal compound, e.g. a ruthenium compound, an iridium compound, or a rhodium compound.

In embodiments, R² and/or R³ in any of the above identified general formulae each independently represent a group selected from alkyl groups having 1 to 10 carbon atoms, aryl groups (e.g. phenyl groups), and cycloalkyl groups having 3 to 8 carbon atoms, wherein the phenyl groups optionally have one or more substituents selected from alkyl groups having 1 to 10 carbon atoms, alkoxy groups having 1 to 10 carbon atoms, and halogen atoms, and each hydrogen atom of the cycloalkyl groups is optionally replaced by an alkyl group having 1 to 10 carbon atoms. Alternatively, R² and R³ in any of the above identified general formulae may form a ring together with carbon atoms to which R² and R³ are bonded, and preferably form a cycloalkane together with the carbon atoms to which R² and R³ are bonded. In any of the above identified general formulae, R² and R³ are each independently preferably a phenyl group (provided that the phenyl group optionally has one or more substituents selected from alkyl groups having 1 to 10 carbon atoms, alkoxy groups having 1 to 10 carbon atoms, and halogen atoms).

The alkyl group having 1 to 10 carbon atoms represented by each of R² and R³ of any of the above identified general formulae can be selected from the groups defined as the alkyl groups having 1 to 10 carbon atoms represented by R¹.

In addition, the aryl group (e.g. phenyl group) represented by each of R² and R³ of any of the above identified general formulae optionally has one or more substituents selected from alkyl groups having 1 to 10 carbon atoms, alkoxy groups having 1 to 10 carbon atoms, and halogen atoms.

The alkyl groups as the substituents can be selected from the above-described groups defined as the alkyl groups represented by R¹ of any of the above identified general formulae.

Each of the alkoxy groups having 1 to 10 carbon atoms as the substituents is preferably a linear or branched alkoxy group having 1 to 5 carbon atoms. Specific examples of the alkoxy group include a methoxy group, an ethoxy group, a n-propoxy group, an isopropoxy group, a n-butoxy group, an isobutoxy group, a s-butoxy group, a t-butoxy group, a n-pentyloxy group, a n-hexyloxy group, a n-heptyloxy group, a n-octyloxy group, a n-nonyloxy group, a n-decyloxy group, and the like. The alkoxy group is preferably a methoxy group, an ethoxy group, a n-propoxy group, an isopropoxy group, a n-butoxy group, an isobutoxy group, a s-butoxy group, a t-butoxy group, or a n-pentyloxy group.

Examples of the halogen atoms as the substituents include chlorine atoms, bromine atoms, fluorine atoms, and the like.

Specific examples of the aryl groups substituted with the substituents represented by each of R² and R³ of any of the above identified general formulae include a 2,4,6-trimethylphenyl group, a 4-methoxyphenyl group, a 2,4,6-trimethoxyphenyl group, a 4-fluorophenyl group, a 2-chlorophenyl group, a 4-chlorophenyl group, a 2,4-dichlorophenyl group, and the like.

The cycloalkyl group having 3 to 8 carbon atoms represented by each of R² and R³ of any of the above identified general formulae is preferably a monocyclic, polycyclic, or bridged cycloalkyl group having 5 to 8 carbon atoms, and particularly preferably a monocyclic cycloalkyl group having 5 to 7 carbon atoms. Specific examples of the cycloalkyl group having 3 to 8 carbon atoms include a cyclopropyl group, a cyclobutyl group, a cyclopentyl group, a cyclohexyl group, a cycloheptyl group, a cyclooctyl group, and the like.

In the cycloalkyl group represented by each of R² and R³ of any of the above identified general formulae, each hydrogen atom is optionally replaced by an alkyl group having 1 to 10 carbon atoms. Specific examples of the alkyl group as the substituent include a methyl group, an isopropyl group, a t-butyl group, and the like.

When R² and R³ of any of the above identified general formulae form a cycloalkane together with carbon atoms to which R² and R³ of any of the above identified general formulae are bonded, R² and R³ of any of the above identified general formulae together form an linear or branched alkylene group having 2 to 10 and preferably 3 to 10 carbon atoms, and form a preferably 4 to 8-membered and more preferably 5 to 8-membered cycloalkane ring, together with the adjacent carbon atoms. Preferred examples of the cycloalkane ring include a cyclopentane ring, a cyclohexane ring, and a cycloheptane ring. In the cycloalkane ring, each hydrogen atom is optionally replaced by an alkyl group having 1 to 10 carbon atoms. Specific examples of the alkyl group as the substituent include a methyl group, an isopropyl group, a t-butyl group, and the like.

In embodiments, Y represents a CH₂ moiety and X represents a group selected from one of an ester (e.g. a t-butyl ester); a thioester; an amide; or a heterocyclic moiety (e.g. a five-membered heterocyclic ring) comprising one or more of O, S, Se, and/or P (e.g. a furan, a tetrahydrofuran, a thiophene, an isoxazole, a bromo-furan, or a thiazole).

A yet further aspect of the invention provides a method for selectively producing optically active compounds using one or more of the metal-diamine complexes (2), (3), (4), or (5) as a catalyst, the method comprising reducing a functional group of a substrate in the presence of one or more of complex (2), (3), (4), or (5) and a hydrogen source or hydrogen donor.

The hydrogen source may be hydrogen gas and/or formic acid, and/or an alcohol and/or a formate salt.

The functional group may be an imine or an imino group, which is reduced to an amine. The functional group may be a ketone or a keto-group, which is reduced to an alcohol.

The functional group may be an unsaturated bond, which is reduced to a saturated bond.

The optically active compounds produced in the method of the invention may be used as precursors for the synthesis of pharmaceutical materials and/or other functional materials.

Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible. For the avoidance of doubt, the terms “may”, “and/or”, “e.g.”, “for example” and any similar term as used herein should be interpreted as non-limiting such that any feature so-described need not be present. Indeed, any combination of optional features is expressly envisaged without departing from the scope of the invention, whether or not these are expressly claimed. The applicant reserves the right to change any originally filed claim or file any new claim accordingly, including the right to amend any originally filed claim to depend from and/or incorporate any feature of any other claim although not originally claimed in that manner.

Embodiments of the invention will now be described by way of example only with reference to the accompanying drawings in which:

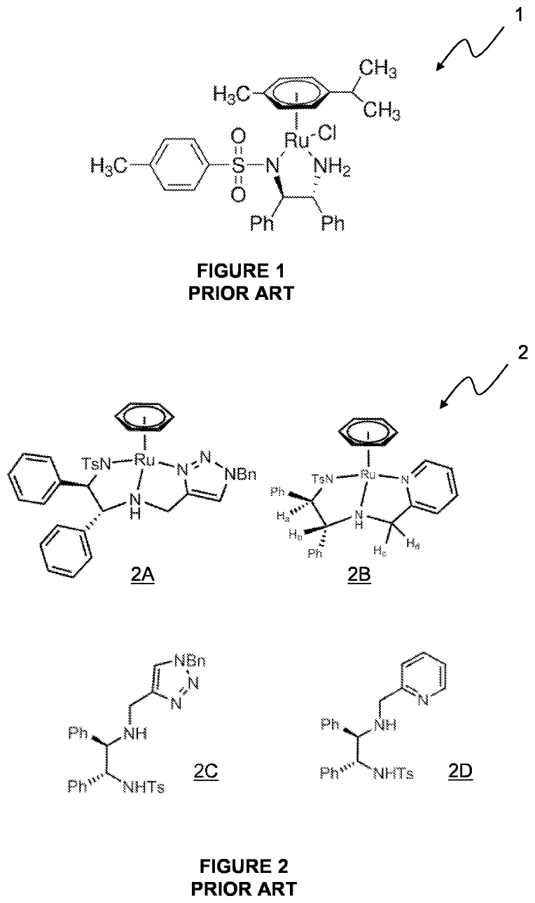

FIG. 1 is a prior art example of a [η⁶-arene)Ru(II)TsDPEN(Cl)] catalyst;

FIG. 2 is a prior art examples of inactive [η⁶-arene)Ru(II)TsDPEN(Cl)] catalysts comprising a tridentate ligand;

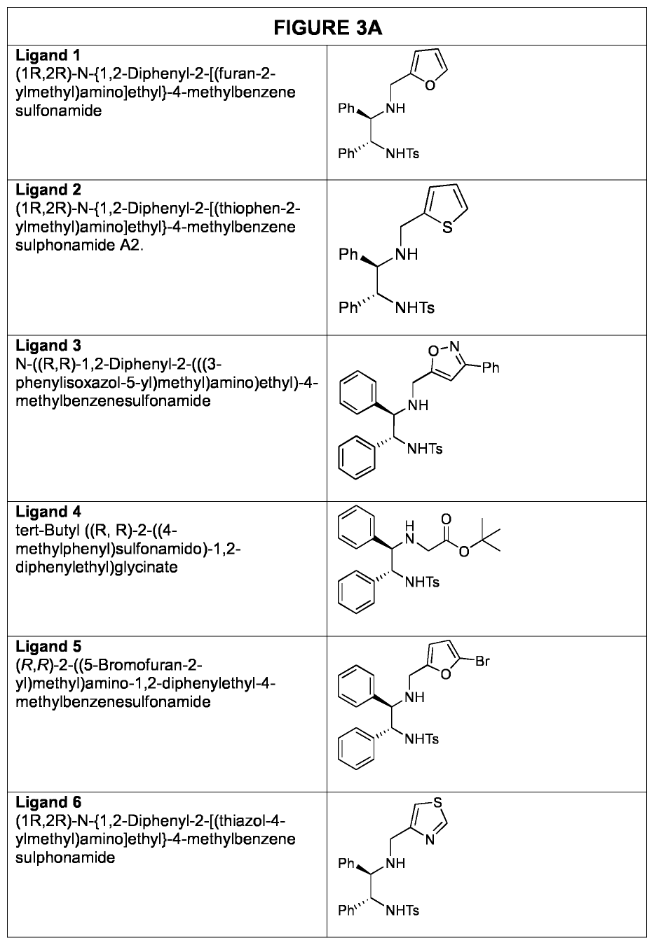

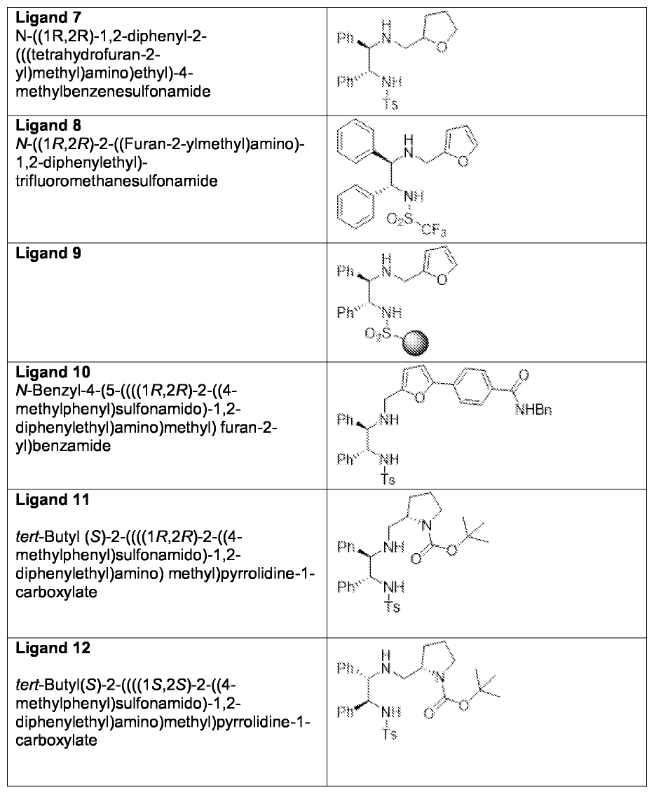

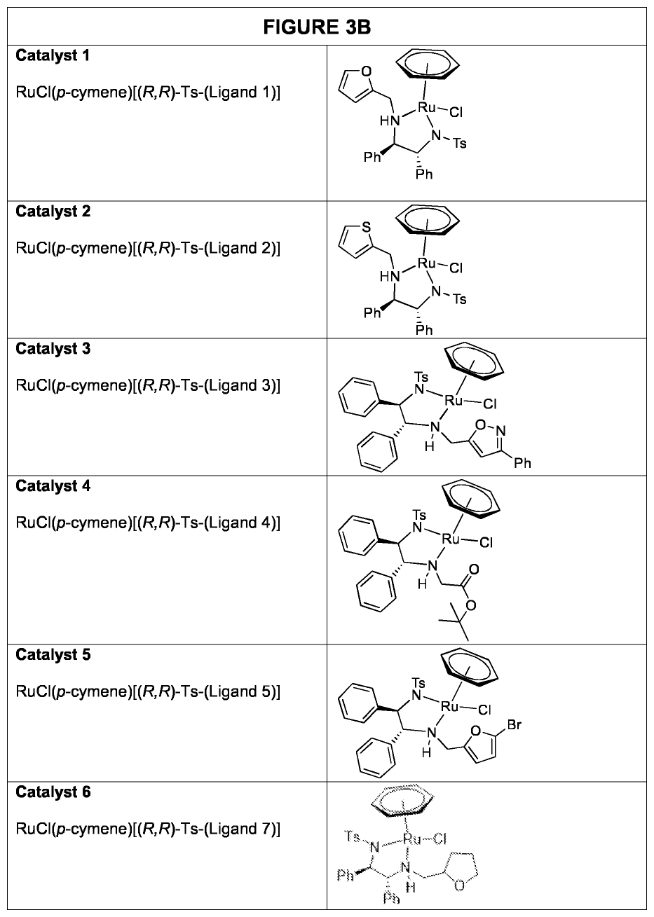

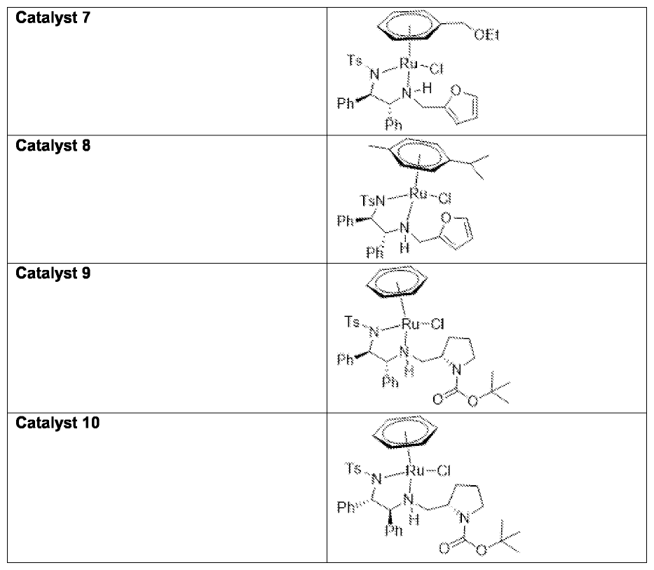

FIG. 3A is a table showing the ligands according to Examples of the invention;

FIG. 3B is a table showing the catalysts according to Examples of the invention.

Referring now to FIG. 1, there is shown an asymmetric catalyst 1 of the prior art. The catalyst 1 is RuCl (p-cymene)[(R,R)-Ts-DPEN], which comprises a TsDPEN ligand with a primary amine.

Referring now to FIG. 2, there is shown inactive [η⁶-arene)Ru(II)TsDPEN(Cl)] catalysts comprising tridentate ligands 2. There is shown a catalyst 2A comprising a ligand 2C which comprises a triazole moiety. There is also shown a catalyst 2B comprising a ligand 2D which comprises a pyridine moiety. Each catalyst 2A and 2B is shown in its inactive form, in which the electron donating triazole moiety (catalyst 2A) or the pyridine moiety (catalyst 2B) is forming a six-membered ring with the ruthenium centre, which has been shown to inhibit the activity of the catalyst. It has been shown that catalyst 2A and catalyst 2B are only active when used with Ru₃(CO)₁₂.

Referring now to FIG. 3A and FIG. 3B, there is shown Ligands 1 to 6 (FIG. 3A) and Catalysts 1 to 5 (FIG. 3B) according to Examples of the invention. It has been surprisingly found that Ligands 1 to 5 may be used to form Catalysts 1 to 5, which are effective catalysts for use in asymmetric transfer hydrogenation reactions in high yield and high enantioselectivity (>90% ee). In contrast, the prior art catalysts shown in FIG. 2 are inactive.

The invention will now be exemplified by way of the following non-limiting Examples.

### Example 1: Synthesis of Ligand 1

### (1R,2R)—N-{1,2-Diphenyl-2-[(furan-2-ylmethyl)amino]ethyl}-4-methylbenzene sulfonamide

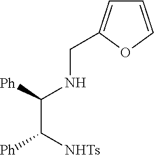

To a solution of (R,R)TsDPEN (500 mg, 1.36 mmol) in dry dichloromethane (20 mL) with 4 Å molecular sieves was added dropwise a solution of 2-furaldehyde (130 mg, 1.36 mmol) in dry dichloromethane (10 mL). The molecular sieves were filtered off after stirring overnight and the solvent removed under reduced pressure. The residue was dissolved in dry THF (8 mL) and cooled to 0° C. followed by dropwise addition of LiAlH₄ (2 M in THF, 0.68 mL, 1.36 mmol). After stirring at rt for 72 h the reaction mixture was cooled to 0° C. and quenched by slow addition of EtOAc. Sat. Rochelle salt solution (15 mL) was added and left to stir at rt for 30 min. Following extraction with EtOAc (3×15 mL), the organic extracts were combined, dried over MgSO₄, filtered and concentrated under reduced pressure to afford the crude product. Purification by column chromatography (10-20% EtOAc in Pet. Ether) afforded the pure product as an off-white solid (523 mg, 1.17 mmol, 86%); Mp 128.4-129.9° C. [α]_{D}²²-41.7 (c 0.05 in CHCl₃), Vₘₐₓ 3298, 3061, 3029, 2916, 2847, 1599, 1505, 1493, 1454, 1373, 1354 cm⁻¹; δ_{H} (500 MHz, CDCl₃) 7.35 (2 H, d, J 8.1, Ts-ArH), 7.30 (1 H, m, 5′-furan), 7.17-7.11 (3 H, m, ArH) 7.10-7.02 (4 H, m, ArH), 7.00 (2 H, d, J 8.1, Ts-ArH), 6.97 (2 H, m, ArH), 6.92 (2 H, t, J 7.0, ArH), 6.26 (1 H, m, 4′-furan), 6.00 (1 H, br. s, NH), 5.98 (1 H, d, J 3.1, 3′-furan), 4.31 (1 H, dd, J 7.3, 2.9 CHNHSO₂), 3.69 (1 H, d, J 7.3, CHNHCH₂), 3.62 (1 H, d, J_{AB} 14.4, CH_{A}H_{B}NH), 3.42 (1 H, d, JAB 14.4, CH_{A}H_{B}NH), 2.32 (3 H, s, Ts-CHs); δ_{C} (125 MHz, CDCl₃) 152.95, 142.70, 141.93, 138.65, 138.29, 137.00, 129.11, 128.41, 127.99, 127.61, 127.58, 127.38, 127.32, 127.08, 110.06, 107.10, 66.51, 62.99, 43.54, 21.43; MS (ESI⁺): m/z, 447.3 [M+H]⁺; HRMS calcd for C₂₆H₂₇N₂O₃S [M+H]⁺ 447.1737, found 447.1736 (0.2 ppm error).

### Example 2: Synthesis of Ligand 2

### (1R,2R)—N-{1,2-Diphenyl-2-[(thiophen-2-ylmethyl)amino]ethyl}-4-methylbenzene sulphonamide

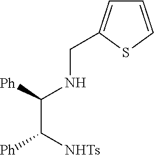

To a solution of (R,R)TsDPEN (400 mg, 1.09 mmol) in dry dichloromethane (15 mL) with 4 Å molecular sieves was added dropwise a solution of 2-thiophenecarboxaldehyde (123 mg, 1.09 mmol) in dry dichloromethane (6 mL). The molecular sieves were filtered off after stirring overnight and the solvent removed under reduced pressure. The residue was dissolved in dry THF (10 mL) and cooled to 0° C. followed by dropwise addition of LiAlH₄ (2 M in THF, 0.55 mL, 1.10 mmol). After stirring at rt overnight the reaction mixture was cooled to 0° C. and quenched by slow addition of EtOAc. Sat. Rochelle salt solution (15 mL) was added and left to stir at rt for 30 min. Following extraction with EtOAc (3×15 mL), the organic extracts were combined, dried over MgSO₄, filtered and concentrated under reduced pressure to afford the crude product. Purification by column chromatography (0-5% MeOH in dichloromethane) afforded the pure product as an off-white solid (388 mg, 0.840 mmol, 77%); Mp 151.4-152.1° C., [α]_{D}²²−31.0 (c 0.05 in CHCl₃), Vₘₐₓ 3336, 3299, 3031, 2930, 2841, 1598, 1492, 1440, 1424, 1347, 1328, 1304 cm⁻¹; δ_{H} (500 MHz, CDCl₃) 7.42 (2 H, d, J 8.1, Ts-ArH), 7.25 (1 H, d, J 5.0, ArH), 7.21-7.16 (3 H, m, ArH) 7.10-7.01 (5 H, m, ArH), 6.99-6.95 (2 H, m, ArH), 6.95-6.90 (3 H, m, ArH), 6.75 (1 H, d, J 2.5, ArH), 6.11 (1 H, d J 2.5, NHSO₂), 4.33 (1 H, dd, J 7.9, 2.6, CHNHSO₂), 3.85 (1 H, d, J_{AB} 14.0, CH_{A}H_{B}NH), 3.75 (1 H, d, J 7.9, CHNHCH₂), 3.64 (1 H, d, J 14.0, CH_{A}H_{B}NH), 2.35 (3 H, s, Ts-CH₃); δ_{C} (125 MHz, CDCl₃) 143.18, 142.76, 138.54, 138.14, 136.99, 129.13, 128.46, 127.95, 127.71, 127.59, 127.53, 127.33, 127.14, 126.63, 124.97, 124.71, 66.37, 62.97, 45.57, 21.45; MS (ESI⁺): m/z, 463.3 [M+H]⁺; HRMS calcd for C₂₆H₂₇N₂O₂S₂ [M+H]⁺ 463.1508, found 463.1511 (0.5 ppm error).

### Example 3: Synthesis of Ligand 3

### N—((R,R)-1,2-Diphenyl-2-(((3-phenylisoxazol-5-yl)methyl)amino)ethyl)-4-methylbenzenesulfonamide

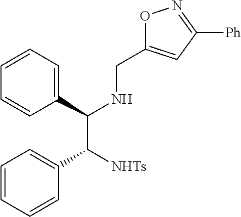

Propargyl/TsDPEN (405 mg, 1.00 mmol) was added to a solution of N-hydroxybenzimidoyl chloride (156 mg, 1.00 mmol) in DMF (2 mL) with 4 Å molecular sieves. After stirring for 96 h, the molecular sieves were filtered off and water (15 mL) was added. The mixture was extracted with EtOAc (3×15 mL) and the combined organic extracts were washed with water (3×15 mL). The organic layer was then dried over MgSO₄, filtered and the solvent removed under reduced pressure to yield the crude product. Purification by column chromatography in (0-30% EtOAc in Pet. Ether) gave the pure product as an off-white solid (416 mg, 0.794 mmol, 79%); [α]_{D}²⁰-43.3 (c 0.05 in CHCl₃); Vₘₐₓ 3244, 3030, 2920, 1727, 1599, 1579, 1494, 1453, 1442, 1407, 1323 cm⁻¹; δ_{H} (500 MHz, CDCl₃) 7.76 (2H, m, ArH), 7.49-7.44 (3H, m, ArH), 7.37 (2H, d, J 8.2, Ts-ArH), 7.19-7.15 (3H, m, ArH), 7.11-7.02 (5H, m, ArH), 7.00 (2 H, d, J 8.2, Ts-ArH) 6.94 (2H, d, J 7.2, ArH), 6.24 (1 H, s, Isoxazole-H), 5.82 (1H, d, J 5.3, NHSO₂), 4.40 (1H, t, J 6.3, CHNHSO₂), 3.87-3.77 (2H, m, CH_{A}H_{B}NH+CHNHCH₂), 3.65 (1H, d, J_{AB}=15.5 Hz, CH_{A}H_{B}NH), 2.31 (3H, s, Ts-CH₃); δ_{C} (126 MHz, CDCl₃) 171.38, 162.31, 142.91, 138.01, 136.84, 130.02, 129.19, 128.98, 128.92, 128.56, 128.17, 127.92, 127.64, 127.53, 127.27, 127.03, 126.82, 99.93, 66.83, 63.11, 42.38, 21.42; MS (ESI⁺): m/z 524.3 [M+H]⁺; HRMS calcd for C₃₁H₃₀N₃O₃S [M+H]⁺ 524.2002, found 524.2003 (0.1 ppm error)

### Example 4: Synthesis of Ligand 4

### tert-Butyl ((R, R)-2-((4-methylphenyl)sulfonamido)-1,2-diphenylethyl)glycinate

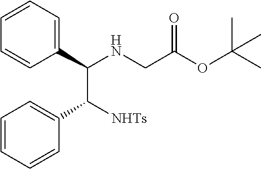

tert-Butyl bromoacetate (205 mg, 1.05 mmol, 155 μL) was added dropwise to a stirring mixture of TsDPEN (366 mg, 1.00 mmol) and K₂CO₃ (207 mg, 1.5 mmol) in MeCN (4 mL). After stirring overnight, the solvent was removed under reduced pressure and the residue dissolved in EtOAc (15 mL) and water (15 mL). The organic layer was separated and the aqueous layer extracted with EtOAC (2×15 mL). The organic extracts were combined, dried over MgSO₄, filtered and the solvent removed under reduced pressure to yield the crude product. Purification by column chromatography (0-30% EtOAc in Pet. Ether) gave the pure product as a white solid (340 mg, 0.708 mmol, 71%); Vₘₐₓ 3250, 3030, 2979, 2929, 1721, 1599, 1494, 1454, 1368, 1346, 1324 Cm⁻¹; δ_{H} (500 MHz, CDCl₃) 7.40 (2 H, d, J 8.1, Ts-ArH), 7.16-7.09 (3 H, m, ArH), 7.08-7.01 (5 H, m, ArH), 6.98-6.94 (2 H, m, ArH), 6.92 (2 H, d, J 6.9, ArH), 6.21 (1 H, m, NHSO₂), 4.32 (1 H, dd, J 7.5, 3.5, CHNHSO₂), 3.72 (1 H, d, J 7.5, CHNHCH₂), 3.14 (1 H, d, J 17.2, CH_{A}H_{B}NH), 3.03 (1 H, d, J 17.2, CH_{A}H_{B}NH), 2.34 (3 H, s, Ts-CH₃), 2.06 (1 H, br. s, NH), 1.41 (9 H, s, 3×CH₃); δ_{C} (126 MHz, CDCl₃) δ ppm 171.14, 142.67, 138.47, 138.28, 137.17, 129.12, 128.31, 127.97, 127.78, 127.68, 127.44, 127.29, 127.08, 81.46, 67.26, 63.13, 49.04, 28.05, 21.44; MS (ESI⁺): m/z 481.3 [M+H]⁺; HRMS calcd for C₂₇H₃₃N₂O₄S [M+H]⁺ 481.2156, found 481.2155 (0.1 ppm error)

### Example 5: Synthesis of Ligand 5

### (R,R)-2-((5-Bromofuran-2-yl)methyl)amino-1,2-diphenylethyl-4-methylbenzenesulfonamide

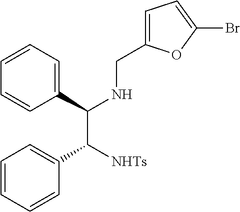

To a solution of (R,R)-TsDPEN (500 mg, 1.36 mmol) in dry dichloromethane (20 mL) with 4 Å molecular sieves was added dropwise a solution 5-bromo-2-furaldehyde (238 mg, 1.36 mmol) in dry dichloromethane (10 mL). The molecular sieves were filtered off after stirring overnight and the solvent removed under reduced pressure. The residue was dissolved in MeOH (15 mL) and NaBH₄ (103 mg, 2.72 mmol) was added portion-wise. The reaction was stirred at room temperature until the imine had reacted completely. Upon completion, the solvent was removed under reduced pressure and the residue partitioned between EtOAc (10 mL) and water (10 mL). The organic fraction collected and extracted again with EtOAc (2×10 mL). Organic extracts were combined, dried over MgSO₄, filtered and concentrated under reduced pressure to afford the crude product. Purification by column chromatography in dichloromethane gave the pure product as an orange solid (368 mg, 0.701 mmol, 52%), [α]_{D}²²−41.7 (c 0.1 in CHCl₃), Vₘₐₓ 3062, 3031, 1597, 1494, 1454, 1326, 1205, 1185, 1155, 1122 cm⁻¹; ¹H NMR (500 MHz, CDCl₃) δ 7.37 (2 H, d, J 8.1, Ts-ArH), 7.18-7.13 (3 H, m, ArH), 7.11-7.04 (3 H, m, ArH), 7.02 (2 H, d, J 8.1, Ts-ArH), 6.99-6.92 (4 H, m, ArH), 6.16 (1 H, d, J 3.1, Furan-2′-H), 5.95 (1 H, d, J 3.1, Furan-3′-H), 5.93 (1 H, d, J 4.4, NHSO₂), 4.33 (1 H, dd, J 7.2, 4.4, CHNHSO₂), 3.71 (1 H, d, J 7.2, CHNHCH₂), 3.59 (1 H, d, J 14.8, CH_{A}H_{B}NH) 3.41 (1 H, d, J 14.8, CH_{A}H_{B}NH) 2.34 (3 H, s, Ts-CH₃), 1.78 (1 H, br. s, NHCH₂); ¹³C NMR (126 MHz, CDCl₃) δ 155.07, 142.76, 138.41, 138.17, 136.95, 129.14, 128.44, 128.06, 127.68, 127.54, 127.42, 127.30, 127.06, 120.77, 111.68, 109.94, 66.48, 62.95, 43.57, 21.44; MS (ESI⁺): m/z 525.2 [M+H]⁺; HRMS calcd for C₂₆H₂₆BrN₂O₃S [M+H]⁺ 525.0842, found 525.0847 (1.0 ppm error).

### Example 6: Synthesis of Ligand 6

### (1R,2R)—N-{1,2-Diphenyl-2-[(thiazol-4-ylmethyl)amino]ethyl}-4-methylbenzene sulphonamide

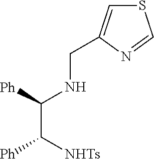

To a solution of (R,R)TsDPEN (366 mg, 1.00 mmol) in dry dichloromethane (15 mL) with 4 Å molecular sieves was added dropwise a solution thiazole-4-carboxaldehyde (113 mg, 1.00 mmol) in dry dichloromethane (5 mL). The molecular sieves were filtered off after stirring overnight and the solvent removed under reduced pressure. The residue was dissolved in dry THF (8 mL) and cooled to 0° C. followed by dropwise addition of LiAlH₄ (2 M in THF, 0.5 mL, 1.00 mmol). After stirring at rt for 5 hours the reaction mixture was cooled to 0° C. and quenched by slow addition of EtOAc. Sat. Rochelle salt solution (12 mL) was added and left to stir at rt for 30 min. Following extraction with EtOAc (3×10 mL), the organic extracts were combined, dried over MgSO₄, filtered and concentrated under reduced pressure to afford the crude product. Purification by column chromatography (3% MeOH in dichloromethane) afforded the pure product as a yellow solid (120 mg, 0.259 mmol, 26%) Mp 67.8-72.4° C., [α]_{D}²²−29.3 (c 0.05 in CHCl₃), Vₘₐₓ 3243, 3062, 3029, 2919, 1598, 1493, 1453, 1409, 1322, 1184, 1153, 1090, 1027; δ_{H} (500 MHz, CDCl₃) 8.75 (1 H, s, 2′-thiazole-H), 7.40 (2 H, d, J 8.2, ArH), 7.18-7.13 (3 H, m, ArH), 7.11-6.98 (9 H, m, ArH), 6.95 (2 H, d, J 7.0, ArH)), 6.28 (1 H, br. s, CH₂NH), 4.38 (1 H, d, J 7.5 PhCHNH), 3.82 (1 H, d, J_{AB} 14.2, CH_{A}H_{B}NH), 3.78 (1 H, d, J 7.5, PhCHNHSO₂), 3.68 (1 H, d, J_{AB} 14.2, CH_{A}H_{B}NH), 2.35 (3 H, s, Ts-CH₃); δ_{C} (125 MHz, CDCl₃) 155.80, 153.01, 142.71, 138.73, 138.31, 137.06, 129.12, 128.39, 127.97, 127.63, 127.45, 127.32, 127.08, 114.46, 67.12, 63.08, 46.80, 21.44; MS (ESI⁺): m/z, 464.2 [M+H]⁺; HRMS calcd for C₂₅H₂₆N₃O₂S₂ [M+H]⁺ 464.1461, found 464.1463 (0.4 ppm error).

### Example 7: Synthesis of Organometallic Catalyst 1 Using Ligand of Example 1

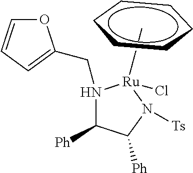

To a Schlenk tube charged with benzene ruthenium (II) chloride dimer (56 mg, 0.112 mmol) and Ligand 1 (100 mg, 0.224 mmol) in dry 2-propanol (6 mL) was added triethylamine (89 mg, 0.880 mmol, 123 μL). After stirring at 80° C. for 1 h, the solution was allowed to cool to rt and the solvent removed under reduced pressure. The residue was dissolved in CHCl₃ (10 mL) and washed with water (10 mL). The organic layer was dried over MgSO₄, filtered and solvent removed under reduced pressure to afford the crude mixture. Purification by column chromatography (15% EtOAc in dichloromethane followed by 1-3% MeOH in dichloromethane) afforded Catalyst 1 as a brown solid (75 mg, 0.114 mmol, 51%); Mp 191.2-194.5° C., [α]_{D}²²+100.0 (c 0.05 in CHCl₃), Vₘₐₓ 3295, 3197, 3061, 3028, 2922, 2871, 1599, 1493, 1453, 1435, 1367, 1329, 1268, 1193, 1148 cm⁻¹; Major diastereomer: δ_{H} (500 MHz, CDCl₃) 7.41 (1 H, s, ArH), 7.33-7.27 (2 H, m, ArH), 7.12-7.02 (3 H, m, ArH) 6.92-6.82 (3 H, m, ArH), 6.77-6.72 (4 H, m, ArH), 6.6 (2 H, d, J 7.3 ArH), 6.37 (2 H, s, ArH), 5.70 (6 H, s, benzene), 4.45 (1 H, br, NH), 4.32-4.24 (1 H, m, CH_{A}H_{B}NH), 4.13 (1 H, d J 13.7, CH_{A}H_{B}NH), 4.05 (1 H, m, PhCHNSO₂), 3.84 (1 H, t, J 11.6, PhCHNH), 2.25 (3 H, s, Ts-CH₃); δ_{C} (125 MHz, CDCl₃) 149.38, 141.92, 141.68, 139.49, 139.41, 136.55, 129.86, 128.93, 128.65, 128.02, 127.56, 126.93, 126.71, 126.48, 111.32, 109.39, 84.01, 80.49, 69.66, 51.52, 21.26; Minor diastereomer: δ_{H} (500 MHz, CDCl₃) 7.49 (1 H, s, ArH), 7.36 (1 H, m, ArH), 7.33-7.27 (2 H, m, ArH) 7.12-7.02 (3 H, m, ArH) 6.82-6.80 (3 H, m, ArH), 6.72-6.69 (4 H, m, ArH), 6.58-6.53 (1 H, m, ArH), 6.41 (1 H, s, ArH), 6.17 (1H, s, ArH), 5.64 (6 H, s, benzene), 5.44 (1H, m, NH), 4.71 (1 H, m, CH_{A}H_{B}NH), 4.39 (1 H, m, PhCHNSO₂), 4.05 (1 H, m, PhCHNH), 3.72 (1 H, d J 14.6, CH_{A}H_{B}NH), 2.23 (3 H, s, Ts-CH₃)); δ_{C} (125 MHz, CDCl₃) 150.46, 143.08, 142.21, 139.39, 138.44, 136.85, 128.60, 128.35, 128.10, 127.53, 126.71, 126.48, 126.41, 125.96, 111.74, 109.16, 83.72, 74.44, 71.86, 50.02, 21.24; MS (ESI⁺): m/z, 625.2 [M−Cl]⁺; HRMS calcd for C₃₂H₃₁N₂O₃SRu [M−Cl]⁺ 625.1093, found 625.1095 (1.0 ppm error).

### Example 8: Synthesis of Organometallic Catalyst 2 Using Ligand of Example 2

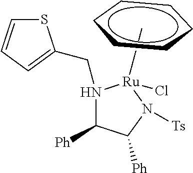

To a Schlenk tube charged with benzene ruthenium (II) chloride dimer (56 mg, 0.112 mmol) and Ligand 2 (100 mg, 0.216 mmol) in dry 2-propanol (6 mL) was added triethylamine (89 mg, 0.880 mmol, 123 μL). After stirring at 80° C. for 1 h, the solution was allowed to cool to rt and the solvent removed under reduced pressure. The residue was dissolved in CHCl₃ (10 mL) and washed with water (10 mL). The organic layer was dried over MgSO₄, filtered and solvent removed under reduced pressure to afford the crude mixture. Purification by column chromatography (20% EtOAc in dichloromethane followed by 1-4% MeOH in dichloromethane) afforded the pure Catalyst 2 as a brown solid (38 mg, 0.056 mmol, 26%) [α]_{D}²²+72.2 (c 0.003 in CHCl₃), Vₘₐₓ 3293, 3062, 3027, 2923, 2868, 1599, 1493, 1453, 1433, 1379, 1328, 1270, 1197 cm⁻¹; δ_{H} (500 MHz, CDCl₃) Major diastereomer: 7.27 (1 H, s, ArH), 7.22 (1 H, m, ArH), 7.14 (1 H, ArH), 7.10-7.04 (2 H, m, ArH), 7.03-6.99 (2 H, m, ArH), 6.95-6.89 (1 H, m, ArH), 6.87-6.79 (3 H, m, ArH), 6.76 (2 H, d, J 7.32, ArH), 6.73-6.69 (2 H, m, ArH), 6.60 (2 H, d, J 7.5, ArH), 5.66 (6 H, s, benzene), 4.54-4.40 (2 H, m, CH_{A}H_{B}NH+CH_{A}H_{B}NH), 4.31 (1 H, d, J 13.7, CH_{A}H_{B}NH), 4.10 (1 H, d, J 10.8, PhCHNSO₂), 3.91-3.80 (1 H, m, PhCHNH), 2.25 (3 H, s, Ts-CH₃); δ_{C} (125 MHz, CDCl₃) 141.94, 139.43, 139.17, 137.93, 136.70, 129.89, 129.05, 128.67, 128.40, 128.01, 127.57, 127.46, 127.05, 126.89, 126.39, 125.09, 84.08, 81.00, 69.59, 54.47, 21.25; MS (ESI⁺): m/z, 641.2 [M−Cl]⁺; HRMS calcd for C₃₂H₃₁N₂O₂S₂Ru [M−Cl]⁺ 641.0865, found 641.0868 (0.7 ppm error).

### Example 9: Synthesis of Organometallic Catalyst 3 Using Ligand of Example 3

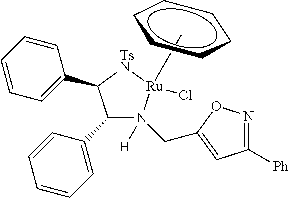

To a Schlenk tube charged with benzene ruthenium (II) chloride dimer (58 mg, 0.116 mmol) and Ligand 3 (120 mg, 0.229 mmol) in PhCl (1.8 mL) was added triethylamine (93 mg, 0.92 mmol, 128 μL). After stirring at 80° C. for 1 h, the solution was allowed to cool to rt and the solvent removed under reduced pressure. The residue was dissolved in CHCl₃ (15 mL) and washed with water (15 mL). The organic layer was dried over MgSO₄, filtered and solvent removed under reduced pressure to afford the crude mixture. Purification by column chromatography (15% EtOAc in dichloromethane followed by 0-3% MeOH in dichloromethane) afforded Catalyst 3 as a dark brown solid (77 mg, 0.104 mmol, 46%) [α]_{D}²²+50.0 (c 0.002 in CHCl₃), Vₘₐₓ 3056, 3029, 1598, 1558, 1493, 1437, 1405, 1374, 1269, 1156, 1127 cm⁻¹; Major: ¹H NMR (500 MHz, CDCl₃) δ 7.84-7.77 (2 H, m ArH), 7.51-7.43 (4 H, m, ArH), 7.33-7.27 (2 H, m, ArH), 7.11-7.06 (3 H, m, ArH), 6.95-6.91 (1 H, m, isoxazole-H), 6.86 (2 H, d, J 7.8, ArH), 6.83-6.78 (2 H, m, ArH), 6.74 (2 H, t, J 7.3, ArH), 6.72-6.67 (1 H, m, ArH), 6.64 (1 H, d, J 7.3, ArH), 5.74 (6 H, s, benzene), 4.58-4.41 (2 H, m, CH_{A}H_{B}NH+CHNSO₂), 4.12 (1 H, d, J 10.68, CH_{A}H_{B}NH), 4.05-3.90 (1 H, m, CHNHCH₂), 2.26 (3 H, s, Ts-CH₃); ¹³C NMR (126 MHz, CDCl₃) 167.24, 163.04, 141.65, 139.65, 139.19, 135.97, 130.53, 129.79, 129.12, 128.94, 128.82, 128.14, 128.09, 127.48, 127.08, 126.99, 126.82, 126.57, 102.60, 84.19, 80.83, 69.36, 50.80, 21.27. MS (ESI⁺): m/z 702.3 [M−Cl]⁺; HRMS calcd for C₃₇H₃₃N₃NaO₃RuS [M−HCl+Na]⁺ 724.1178, found 724.1188 (0.0 ppm error)

### Example 10: Synthesis of Organometallic Catalyst 4 Using Ligand of Example 4

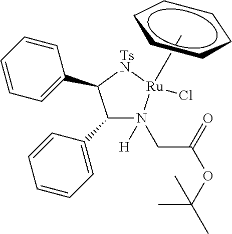

To a Schlenk tube charged with benzene ruthenium (II) chloride dimer (52 mg, 0.104 mmol) and Ligand 4 (100 mg, 0.208 mmol) in PhCl (1.5 mL) was added triethylamine (84 mg, 0.832 mmol, 116 μL). After stirring at 80° C. for 1 h, the solution was allowed to cool to rt and the solvent removed under reduced pressure. The residue was dissolved in CHCl₃ (10 mL) and washed with water (15 mL). The organic layer was dried over MgSO₄, filtered and solvent removed under reduced pressure to afford the crude mixture. Purification by column chromatography (10% EtOAc in dichloromethane followed by 0-3% MeOH in dichloromethane) afforded Catalyst 4 as a dark brown solid (87 mg, 0.125 mmol, 60%) [α]_{D}²²+631.3 (c 0.002 in CHCl₃); Vₘₐₓ 3060, 3029, 2978, 2931, 1731, 1636, 1600, 1494, 1454, 1436, 1393 cm⁻¹; Major: ¹H NMR (500 MHz, CDCl₃) δ ppm 7.32 (2 H, d, J 8.1, Ts-ArH), 7.16-7.05 (4 H, m, ArH), 6.82 (2 H, d, J 7.9, ArH), 6.79-6.75 (1 H, m, ArH), 6.74-6.68 (3 H, m, ArH), 6.64-6.54 (2 H, m, ArH), 6.06-5.94 (1 H, dd, J 11.4, 6.6, NH), 5.83 (6 H, s, benzene), 4.41 (1 H, dd, J 18.4, 6.6, CH_{A}H_{B}NH), 4.32 (1 H, d, J 10.8, CHNSO₂), 4.13 (1 H, t, J 11.6, CHNHCH₂), 3.27 (1 H, d, J 18.4, CH_{A}H_{B}NH), 2.23 (3 H, s, Ts-CH₃), 1.44 (9 H, s, 3×CH₃). ¹³C NMR (126 MHz, CDCl₃) δ ppm 170.65, 143.13, 139.40, 138.66, 135.46, 129.82, 128.55, 128.47, 128.44, 128.08, 126.51, 126.49, 125.92, 84.08, 84.03, 73.79, 71.53, 56.57, 27.85, 21.23; MS (ESI⁺): m/z 659.3 [M−Cl]⁺; HRMS calcd for C₃₃H₃₇N₂O₄RuS [M−Cl]⁺ 659.1512, found 659.1526 (0.8 ppm error).

### Example 11: Synthesis of Organometallic Catalyst 5 Using Ligand of Example 5

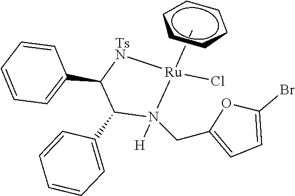

To a Schlenk tube charged with benzene ruthenium (II) chloride dimer (40 mg, 0.081 mmol) and Ligand 5 (85 mg, 0.162 mmol) in PhCl (1.2 mL) was added triethylamine (66 mg, 0.65 mmol, 90 μL). After stirring at 85° C. for 1 h, the solution was allowed to cool to rt and the solvent removed under reduced pressure. The residue was dissolved in CHCl₃ (10 mL) and washed with water (12 mL). The organic layer was dried over MgSO₄, filtered and solvent removed under reduced pressure to afford the crude mixture. Purification by column chromatography (15% EtOAc in dichloromethane followed by 0-3% MeOH in dichloromethane) afforded the Catalyst 5 as a brown solid (85 mg, 0.115 mmol, 71%); [α]_{D}²²+100.0 (c 0.05 in CHCl₃), Vₘₐₓ 3062, 3027, 2919, 2871, 1599, 1494, 1454, 1436, 1398, 1374 cm⁻¹; Major diastereomer; ¹H NMR (500 MHz, CDCl₃) δ ppm 7.34-7.28 (2 H, m, ArH), 7.13-7.03 (4 H, m, ArH), 6.90-6.79 (3 H, m, ArH), 6.78-6.66 (3 H, m, ArH), 6.60 (2 H, d, J 7.9 Ts-ArH), 6.36 (1 H, m, 3′furan-H), 6.25 (1 H, d, J 3.20, 2′furan-H), 5.78 (6 H, s, benzene-H), 4.38-4.33 (1 H, m, CH_{A}H_{B}NH), 4.16-4.11 (1 H, m, CH_{A}H_{B}NH), 4.04 (1 H, d, J 10.8, CHNHCH₂), 3.86-3.81 (1 H, m, CHNSO₂), 2.25 (3 H, s, Ts-ArH); ¹³C NMR (126 MHz, CDCl₃) δ ppm 151.24, 141.57, 139.57, 139.36, 136.30, 129.79, 128.83, 128.63, 128.43, 128.04, 127.55, 126.98, 126.49, 121.33, 112.82, 112.44, 84.17, 80.56, 69.67, 51.60, 21.25; MS (ESI⁺): m/z 703.1 [M+H]⁺; HRMS calcd for C₃₂H₃₀BrN₂O₃RuS [M−Cl]⁺ 703.0199, found 703.0202 (0.1 ppm error)

### Example 12: Synthesis of Ligand 7

### N-((1R,2R)-1,2-diphenyl-2-(((tetrahydrofuran-2-yl)methyl)amino)ethyl)-4-methylbenzenesulfonamide

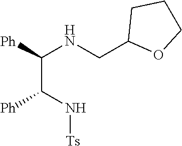 [SMILES: Cc1ccc(S(=O)(=O)N[C@H](c2ccccc2)[C@H](NCC2CCCO2)c2ccccc2)cc1]

dr 1:1

To a mixture of R,R-TsDPEN (366 mg, 1.0 mmol) and 4 Å molecular sieves in DCM (8 mL) was added dropwise a solution of tetrahydrofuran-2-carbaldehyde (100 mg, 1.0 mmol) in DCM (5 mL). The molecular sieves were filtered off after stirring overnight and the solvent was removed under reduced pressure. The residue was dissolved in MeOH (10 mL) and acetic acid was added (6 drops) followed by slow addition of NaBH₃CN (76 mg, 1.2 mmol). After stirring overnight, the solvent was removed under reduced pressure and the residue was partitioned in EtOAc (15 mL) and H₂O (15 mL). The organic layer was collected and further extracted with EtOAc (2×15 mL). The organic extracts were combined, dried over MgSO₄, filtered and the solvent removed under reduced pressure. Purification by column chromatography (0-20% EtOAc in Pet. Ether) gave Ligand 7 as a white solid (265 mg, 0.59 mmol, 59%); [α]_{D}−12.0 (c 0.2 in CHCl₃); ¹H NMR (500 MHz, CDCl₃) δ 7.41-7.35 (4H, m, ArH), 7.14-7.10 (6H, m, ArH), 7.07-6.99 (12H, m, ArH), 6.97-6.92 (5H, m, ArH), 6.91-6.87 (4H, m, ArH), 4.29 (1H, d, J 7.6), 4.20 (1H, d, J 8.3), 4.01-3.93 (1H, m), 3.90-3.84 (1H, m), 3.80-3.74 (1H, m), 3.73-3.67 (4H, m), 3.62 (1 H, d, J 8.3), 2.53 (1H, d, J 12.1), 2.39 (2H, d, J 5.8), 2.34 (3H, s, Ts-CH₃), 2.33 (3H, s, Ts-CH₃), 1.95-1.88 (2H, m), 1.87-1.76 (6H, m), 1.59-1.50 (1H, m), 1.48-1.39 (1H, m); ¹³C NMR (126 MHz, CDCl₃) δ 142.66, 142.65, 139.21, 139.11, 138.43, 138.18, 137.17, 137.07, 129.09, 129.08, 128.31, 128.27, 127.92, 127.81, 127.72, 127.55, 127.50, 127.45, 127.25, 127.24, 127.19, 127.09, 78.46, 78.12, 68.11, 68.06, 68.01, 67.79, 63.25, 63.07, 51.54, 51.02, 29.04, 29.02, 25.88, 25.72, 21.44; MS (ESI⁺) m/z 451.3 [M−H]⁺; HRMS calcd for C₂₆H₃₁N₂O₃S [M−H]⁺ 451.2050, found 451.2053 (0.7 ppm err).

### Example 13: Synthesis of Ligand 8

### N-((1R,2R)-24(Furan-2-ylmethyl)amino)-1,2-diphenylethyl)-trifluoromethanesulfonamide

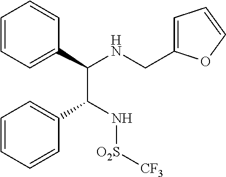 [SMILES: O=S(=O)(N[C@H](c1ccccc1)[C@H](NCc1ccco1)c1ccccc1)C(F)(F)F]

To a mixture of R,R-TsDPEN (424 mg, 2.0 mmol) and 4 Å molecular sieves in DCM (20 mL) was added dropwise a solution of 2-furaldehyde (192 mg, 2.0 mmol) in DCM (10 mL). The molecular sieves were filtered off after stirring overnight and the solvent was removed under reduced pressure. The residue was dissolved in MeOH (15 mL) and acetic acid was added (6 drops) followed by slow addition of NaBH₃CN (190 mg, 3 mmol). After stirring overnight, the solvent was removed under reduced pressure and the residue was partitioned in EtOAc (15 mL) and H₂O (15 mL). The organic layer was collected and further extracted with EtOAc (2×15 mL). The organic extracts were combined, dried over MgSO₄, filtered and the solvent removed under reduced pressure. Purification by column chromatography (0-100% EtOAc in Pet. Ether) gave the precursor (1R,2R)-N1-(furan-2-ylmethyl)-1,2-diphenylethane-1,2-diamine as an orange oil (290 mg, 1.0 mmol, 50%); vₘₐₓ 3060, 3027, 2828, 1681, 1600, 1493, 1452, 1346 cm⁻¹; ¹H NMR (500 MHz, CDCl₃) δ 7.33-7.29 (1H, m, ArH), 7.25-7.19 (4H, m, ArH), 7.18-7.13 (5H, m, ArH), 7.13-7.08 (2H, d, J 7.1 Hz, ArH), 6.31-6.23 (1 H, m, ArH), 6.00 (1H, d, J 2.8 Hz, ArH), 3.99 (1H, d, J 7.2 Hz, CHPh), 3.74 (1 H, d, J 7.2 Hz, CHPh), 3.67 (1 H, d, J 14.7 Hz, CH_{A}H_{B}), 3.51-3.42 (1 H, m, CH_{A}H_{B}), 1.85 (2H, b.s, NH₂); ¹³C NMR (126 MHz, CDCl₃) δ 154.16, 143.58, 141.81, 140.89, 128.43, 128.25, 128.23, 128.12, 127.26, 127.07, 110.11, 106.85, 68.56, 61.89, 44.06; MS (ESI⁺) m/z 293.2 [M−H]⁺

To a solution of (1R,2R)-N1-(furan-2-ylmethyl)-1,2-diphenylethane-1,2-diamine (50 mg, 0.17 mmol) in DCM (3 mL) was added triethylamine (100 μL, 0.17 mmol). The mixture was cooled to 0° C. and Tf₂O (1M in DCM, 0.17 mL, 0.17 mmol) was added dropwise. After stirring overnight, the mixture was diluted with DCM (10 mL) and quenched with sat. NaHCO₃ solution (10 mL). Following extraction with DCM (2×10 mL) the organic extracts were combined, dried over MgSO₄, filtered and the solvent removed under reduced pressure. Purification by column chromatography (10-30% EtOAc) gave Ligand 8 as an orange semi-solid (43 mg, 0.10 mmol, 59%); [α]_{D}−17.3 (c 0.1 in CHCl₃); vₘₐₓ 3064, 3034, 2970, 1603, 1496, 1456, 1380 cm⁻¹; ¹H NMR (500 MHz, CDCl₃) δ 7.39-7.30 (5H, m, ArH), 7.30-7.26 (2H, m, ArH), 7.26-7.17 (4H, m, ArH), 6.29-6.23 (1H, m, Furan-H), 5.96 (1H, d, J 3.0, Furan-H), 4.70 (1H, d, J 5.1, PhCHNTf), 3.94 (1H, d, J 5.1, NCHPh), 3.68 (d, J 14.8, CH_{A}H_{B}), 3.47 (1H, d, J 14.8, CH_{A}H_{B}); ¹³C NMR (126 MHz, CDCl₃) δ 152.56, 142.26, 138.28, 138.09, 128.95, 128.85, 128.42, 128.32, 127.64, 126.54, 119.31 (q, J321, CF₃), 110.23, 107.53, 66.21, 64.02, 43.55. ¹⁹F NMR (282 MHz, CDCl₃) δ−77.45; MS (ESI⁺) m/z 425.2 [M+H]⁺; HRMS calcd for C₂₀H₂₀F₃N₂O₃S [M+H]⁺ 425.1141, found 425.1143 (0.4 ppm error).

### Example 14: Synthesis of Ligand 9

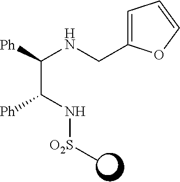 [SMILES: CS(=O)(=O)N[C@H](c1ccccc1)[C@H](NCc1ccco1)c1ccccc1]

To a suspension of sulfonyl chloride polymer bound (85 mg, 1.5-2.0 mmol/g) and triethylamine (0.1 mL, 0.17 mmol) in DCM (3 mL) was added (1R,2R)-N1-(furan-2-ylmethyl)-1,2-diphenylethane-1,2-diamine (the precursor to Ligand 8) (50 mg, 0.17 mmol). After gentle stirring overnight, the product was filtered off and washed with DCM (3×3 mL) and water (3×3 mL). The precipitate was dried to give Ligand 9a as an off-white solid (103 mg).

A supported catalyst could also be prepared using a silica support in which case the procedure would be; to a suspension of 3-chloropropyl silica (100 mg, 1.3-2.1 mmol/g) in methanol (10 mL) was added (1R,2R)-N1-(furan-2-ylmethyl)-1,2-diphenylethane-1,2-diamine (the precursor to Ligand 8) (50 mg, 0.17 mmol). After gentle stirring overnight, the product was filtered off and washed with methanol (5×10 mL) and water (5×10 mL). The solid was dried to give Ligand 9b as off-white particles. Note: the structure of this would not contain a sulfonyl group.

### Example 15: Synthesis of Ligand 10

### N-Benzyl-4-(5-((((1R,2R)-2-((4-methylphenyl)sulfonamido)-1,2-diphenylethyl)amino)methyl) furan-2-yl)benzamide

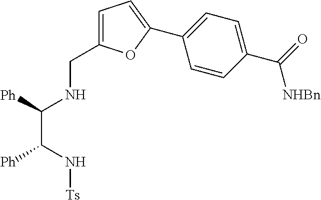

To a mixture of (R,R)-TsDPEN (50 mg, 0.14 mmol) and 4-(5-formylfuran-2-yl)benzoic acid (30 mg, 0.14 mmol) in EtOH (5 mL) was added 4 drops of acetic acid. After refluxing for 4 h, the solution was cooled to rt and the solvent removed under reduced pressure. DCM (5 mL) was added to the residue and the solution was filtered. The precipitate was washed again with DCM (2×5 mL). The filtrates were combined and the solvent was removed under reduced pressure to give the crude product. Purification by column chromatography (50% EtOAc in Pet. Ether) afforded 4-(5-((4R,5R)-4,5-diphenyl-1-tosylimidazolidin-2-yl)furan-2-yl)benzoic acid as an orange solid (52 mg, 0.092 mmol, 66%); 1H NMR (400 MHz, CDCl₃) δ 8.12 (2H, d, J 8.4, ArH), 7.67 (2H, d, J 8.4, ArH), 7.46 (2H, d, J 8.2, ArH), 7.30-7.27 (4H, m, ArH), 7.25-7.20 (3H, m, ArH), 7.17-7.12 (3H, m, ArH), 7.10 (2H, d, J 8.2, ArH), 6.84 (1H, d, J 3.4, Furan-H), 6.68 (1H, d, J 3.4, Furan-H), 6.19 (1H, s, NCHN), 4.88 (1H, d, J 6.8, CHPh), 4.55 (1H, d, J 6.8, CHPh), 2.33 (3H, s, Ts-CH₃); MS (ESI+): m/z 565.3 [M+H]+; HRMS calcd for C₃₃H₂₉N₂O₅S [M−Cl]⁺565.1792, found 565.1794 (0.4 ppm error).

To a mixture of 4-(5-((4R,5R)-4,5-diphenyl-1-tosylimidazolidin-2-yl)furan-2-yl)benzoic acid (100 mg, 177 μmol), benzylamine (21 mg, 195 μmol) and DMAP (32 mg, 266 μmol) in DCM (5 mL) was added EDCl (44 mg, 230 μmol). After stirring at rt for 72 h, the reaction mixture was quenched with water (10 mL). Following extraction with DCM (3×10 mL), the organic fractions were combined, dried over MgSO₄, filtered and the solvent removed under reduced pressure. Purification by column chromatography (gradient elution 0-50% EtOAc in Pet. Ether) gave N-Benzyl-4-(5-((4R,5R)-4,5-diphenyl-1-tosylimidazolidin-2-yl)furan-2-yl)benzamide as yellow solid (83 mg, 127 μmol, 72%); vₘₐₓ 3290, 3034, 2921, 1639, 1535, 1494, 1453 cm⁻¹; ¹H NMR (300 MHz, CDCl₃) δ 7.88 (2H, s, ArH), 7.84-7.78 (2H, m, ArH), 7.64 (2H, d, J 8.3, ArH), 7.45 (1H, d, J 8.2, ArH), 7.41-7.36 (6H, m, ArH), 7.35-7.31 (2H, m, ArH), 7.24-7.18 (4H, m, ArH), 7.15-7.11 (2H, m, ArH), 7.08 (2H, J 8.3, ArH), 6.76 (1H, d, J 3.3, Furan-H), 6.65 (1H, d, J 3.3, Furan-H), 6.43 (2H, b.s NH×2), 6.16 (1H, s, NCHNTs), 4.85 (1H, d, J 6.7, CHPh), 4.71-4.63 (2H, m, NCH₂Ph), 4.54 (1H, d, J 6.7, CHPh), 2.32 (3H, s, Ts-CH₃); MS (ESI) [M−H]⁻ 652.2, [M+H]⁺ 654.3.

### N-Benzyl-4-(5-((4R,5R)-4,5-diphenyl-1-tosylimidazolidin-2-yl)furan-2-yl)benzamide

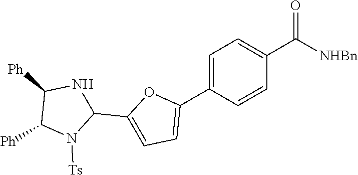

N-Benzyl-4-(5-((4R,5R)-4,5-diphenyl-1-tosylimidazolidin-2-yl)furan-2-yl)benzamide (JB382) (50 mg, 76 μmol) was dissolved in MeOH (1.5 mL) and acetic acid was added (3 drops) followed by slow addition of NaBH₃CN (8 mg, 129 μmol). After stirring overnight, the solvent was removed under reduced pressure and the residue was partitioned in EtOAc (10 mL) and H₂O (10 mL). The organic layer was collected and further extracted with EtOAc (2×15 mL). The organic extracts were combined, dried over MgSO₄, filtered and the solvent removed under reduced pressure. Purification by column chromatography (0-50% EtOAc in Pet. Ether) gave Ligand 10 as an orange solid (32 mg, 49 μmol, 64%); [α]_{D}−63.1 (c 0.2 in CHCl₃); vₘₐₓ 2986, 2926, 2870, 2030, 1680, 1667 cm⁻¹; ¹H NMR (500 MHz, CDCl₃) δ 7.81 (2H, d, J 8.4, ArH), 7.61 (2H, d, J 7.7, ArH), 7.40-7.34 (5H, m, ArH), 7.32 (3H, d, J 8.2, ArH), 7.17-7.13 (3H, m, ArH), 7.06-6.98 (5H, m, ArH), 6.96 (2H, d, J 8.2, ArH), 6.93 (2H, d, J 7.2, ArH+NHSO₂), 6.61 (1 H, d, J 3.2, Furan-H), 6.49-6.42 (1H, m, C(O)NH), 6.09 (1H, d, J 3.2, Furan-H), 4.67 (2H, d, J 5.5, C(O)NHCH₂), 4.35 (1 H, d, J 7.3, CHPh), 3.77-3.70 (2H, m, CHPh+CH_{A}CH_{B}), 3.51 (1H, d, J 14.8, CH_{A}CH_{B}), 2.30 (3H, s, Ts-CH₃), 1.85 (1H, bs, NH); ¹³C NMR (126 MHz, CDCl₃) δ 166.99, 153.74, 152.42, 142.85, 138.60, 138.32, 137.07, 133.68, 132.64, 129.23, 128.96, 128.59, 128.19, 128.15, 127.82, 127.73, 127.61, 127.52, 127.40, 127.15, 123.65, 109.81, 107.45, 66.54, 63.11, 44.33, 43.74, 21.54; MS (ESI⁺) m/z 656.4 [M+H]⁺; HRMS calcd for C₄₀H₃₈N₃O₄S [M+H]⁺ 656.2578, found 656.2574 (0.5 ppm error)

This ligand may undergo further reaction to bond a solid support, e.g. a polymeric or silica particle, thereto via the amide moiety.

### Example 16: Synthesis of Organometallic Catalyst 6 Using Ligand 7 of Example 12

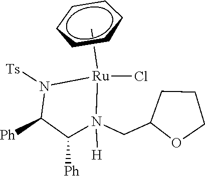

To a Schlenk tube charged with benzene ruthenium (II) chloride dimer (56 mg, 0.11 mmol) and Ligand 7 of example 12 (100 mg, 0.22 mmol) in PhCl (1 mL) was added triethylamine (90 mg, 0.89 mmol, 120 μL). After stirring at 85° C. for 1 h, the solution was allowed to cool to rt and the solvent removed under reduced pressure. The residue was dissolved in CHCl₃ (10 mL) and washed with water (10 mL). The organic layer was dried over MgSO₄, filtered and solvent removed under reduced pressure to afford the crude mixture. Purification by column chromatography (15% EtOAc in dichloromethane followed by 0-3% MeOH in dichloromethane) afforded Catalyst 6 as a brown solid (19 mg, 0.029 mmol, 13%); MS (ESI⁺): m/z 629.3 [M−Cl]+; HRMS calcd for C₃₂H₃₅N₂O₃RuS [M−Cl]+629.1406, found 629.1417 (0.4 ppm error).

### Example 17: Synthesis of Organometallic Catalyst 7 Using Ligand 1 of Example 1

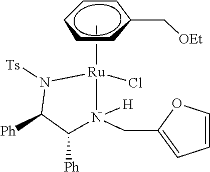

dr 3:2

To a solution of RuCl₃XH₂O (91 mg, 0.44 mmol) in EtOH (4 mL) was added a solution of cyclohexa-1,4-dien-1-ylmethanol (132 mg, 0.96 mmol) in EtOH (1 mL). After refluxing for 16 h, the mixture was cooled to rt and the precipitate was filtered. The precipitate was washed with ice-cold EtOH (3×2 ml) and then dried to afford the ruthenium dimer as a black solid (58 mg, 0.19 mmol, 43%); vₘₐₓ 3064, 3057, 2967, 2922, 2863, 1443, 1397 cm⁻¹; ¹H NMR (400 MHz, CDCl₃) δ 5.77-5.51 (5H, m, ArH, 4.46 (2H, s, ArCH₂), 3.67 (2H, m, 2H), 1.23 (3H, t, 6.5 Hz, CH₃). To a Schlenk tube charged with ruthenium dimer (25 mg, 41 μmol) and Ligand 1 (36 mg, 81 μmol) in PhCl (1 mL) was added triethylamine (32 mg, 81 μmol, 45 μL). After stirring at 80° C. for 1 h, the solution was allowed to cool to rt and the solvent removed under reduced pressure. The residue was dissolved in CHCl₃ (10 mL) and washed with water (10 mL). The organic layer was dried over MgSO₄, filtered and the solvent removed under reduced pressure to afford the crude mixture. Purification by column chromatography (10% EtOAc in DCM followed by 0-3% MeOH in DCM) afforded Catalyst 7 as a brown solid (17 mg, 24 μmol, 30%); vₘₐₓ 3675, 2973, 2901, 1494, 1453, 1394, 1381 cm⁻¹; ¹H NMR (500 MHz, CDCl₃) δ 7.42 (1 H, s, ArH), 7.18 (2H, d, J 8.0, ArH), 7.12-7.00 (3H, m, ArH), 6.83 (2H, d, J 7.7, ArH), 6.75-6.69 (4H, m, ArH), 6.59-6.56 (2H, m, ArH), 6.47 (2H, t, J 7.7, ArH), 6.38-6.35 (1 H, m, ArH), 6.12 (1 H, d, J 3.0, ArH), 6.06 (1H, t, J 5.7, Arene-H), 5.90-5.86 (1H, m, Arene-H), 5.77-5.71 (1H, m, Arene-H), 5.26 (1H, t, J 5.7, Arene-H), 5.15-5.10 (1H, m, Arene-H), 4.73-4.67 (2H, m, ArCH₂O), 4.54 (1H, d, J 11.5, PhCHNTs), 4.30 (1H, d, J 11.5, PhCHNH), 3.90-3.85 (1 H, m, OCH₂), 3.83-3.77 (1H, m, OCH₂), 3.74 (1H, t, J 11.5, NHCH₂), 3.66 (1H, d, J 14.5, NHCH₂), 2.20 (3H, s, Ts-CHs), 1.47 (3H, t, J 7.0, OCH₂CH₃); ¹³C NMR (126 MHz, CDCl₃) δ 150.82, 143.47, 142.15, 139.08, 137.56, 137.40, 130.21, 129.13, 128.73, 128.39, 128.18, 128.05, 126.87, 126.45, 111.45, 110.30, 93.23, 89.14, 87.16, 86.42, 82.86, 79.92, 77.96, 75.19, 72.23, 70.60, 68.21, 49.57, 21.32, 15.18; MS (ESI⁺) M/Z 683.3[M−Cl]⁺; HRMS calcd for C₃₅H₃₇N₂O₄RuS [M−Cl]⁺ 683.1512, found 683.1517 (0.7 ppm error).

### Example 18: Synthesis of Organometallic Catalyst 8 Using Ligand 1 of Example 1

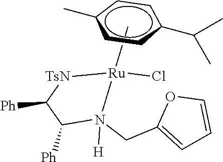

dr 2:1

To a Schlenk tube charged with p-cymene ruthenium (II) chloride dimer (55 mg, 0.09 mmol) and Ligand 1 (80 mg, 0.18 mmol) in PhCl (1.25 mL) was added triethylamine (73 mg, 0.72 mmol, 0.1 mL). After stirring at 80° C. for 1 h, the solution was allowed to cool to rt and the solvent removed under reduced pressure. The residue was dissolved in CHCl₃ (10 mL) and washed with water (10 mL). The organic layer was dried over MgSO₄, filtered and the solvent removed under reduced pressure to afford the crude mixture. Purification by column chromatography (15% EtOAc in DCM followed by 0-3% MeOH in DCM) afforded Catalyst 8 as a brown solid (74 mg, 0.103 mmol, 57%); vₘₐₓ 3194, 3062, 3029, 2961, 2922, 2867, 1599, 1494, 1452, 1383 cm⁻¹; ¹H NMR (500 MHz, CDCl₃) δ 7.41 (1H, s, ArH), 7.21-7.17 (2H, m, ArH), 7.10-7.05 (3H, m, ArH), 6.78 (2H, d, J 8.0, ArH), 6.74 (1H, d, J 8.0, ArH), 6.67 (2H, t, J 7.6, ArH), 6.61 (1H, d, J 7.6, ArH), 6.53 (2H, d, J 7.2, ArH), 6.48 (1H, t, J 7.6, ArH), 6.42-6.38 (2H, m, ArH), 5.54 (1H, b.s, p-Cymene ArH), 5.41 (2H, t, J 5.7, p-Cymene ArH), 5.18 (1H, b.s, p-cymene ArH), 4.56 (1H, t, J 10.2, NH), 4.20 (1H, dd, J 14.8, 10.2, CH_{A}H_{B}N), 4.08 (1H, d, J 11.5, PhCHNTs), 4.03 (1H, d, J 14.8, CH_{A}H_{B}N), 3.63 (1H, t, J 11.5, PhCHNH), 3.28-3.17 (1H, m, p-cymene CH(CH₃)₂, 2.32 (3H, s, p-cymene ArCH₃), 2.22 (3H, s, Ts-CH₃), 1.40 (3H, d, J 7.0, p-cymene CH(CH₃)₂), 1.33 (3H, d, J 7.0, p-cymene CH(CH₃)₂); ¹³C NMR (126 MHz, CDCl₃) δ 149.72, 142.41, 141.77, 139.23, 138.96, 136.89, 129.23, 128.72, 128.34, 127.96, 127.48, 127.03, 126.53, 126.37, 111.46, 109.02, 81.50, 79.73, 78.89, 75.25, 72.21, 70.23, 60.53, 53.57, 51.44, 30.62, 22.59, 22.38, 21.33, 19.19; MS (ESI⁺): m/z 681.3 [M−Cl]⁺; HRMS calcd for C₃₆H₃₉N₂O₃RuS [M−Cl]⁺ 681.1719, found 681.1728 (0.1 ppm error)

### Example 19: Synthesis of Ligand 11

### tert-Butyl (S)-2-((((1R,2R)-2-((4-methylphenyl)sulfonamido)-1,2-diphenylethyl)amino) methyl)pyrrolidine-1-carboxylate

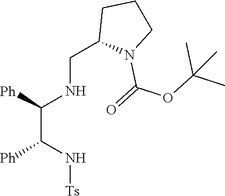 [SMILES: Cc1ccc(S(=O)(=O)N[C@H](c2ccccc2)[C@H](NC[C@@H]2CCCN2C(=O)OC(C)(C)C)c2ccccc2)cc1]

To a mixture of R,R-TsDPEN (366 mg, 1.0 mmol) and 4 Å molecular sieves in DCM (10 mL) was added dropwise a solution of (S)-Boc-prolinal (199 mg, 1.0 mmol) in DCM (5 mL). The molecular sieves were filtered off after stirring overnight and the solvent was removed under reduced pressure. The residue was dissolved in MeOH (10 mL) and acetic acid was added (6 drops) followed by slow addition of NaBH₃CN (76 mg, 1.2 mmol). After stirring overnight, the solvent was removed under reduced pressure and the residue was partitioned in EtOAc (15 mL) and H₂O (15 mL). The organic layer was collected and further extracted with EtOAc (2×15 mL). The organic extracts were combined, dried over MgSO₄, filtered and the solvent removed under reduced pressure. Purification by column chromatography (0-30% EtOAc in Pet. Ether) gave Ligand 11 as a white solid, which forms rotamers (471 mg, 0.86 mmol, 86%); [α]_{D}−41.2 (c 0.3 in CHCl₃); vₘₐₓ 3262, 3064, 3030, 2973, 2929, 2874, 1687, 1600, 1494, 1477 cm⁻¹: ¹H NMR (500 MHz, CDCl₃) δ 7.36 (2H, d, J 7.6, ArH), 7.12 (3H, b. s., ArH), 7.02 (5H, b.s., ArH), 6.91 (4H, b. s, ArH), 4.35-4.16 (1H, m, PhCHNTs), 3.93-3.63 (1H, m, PhCHNH), 3.64-3.33 (1H, m, Pyrrolidine-CH), 3.32-3.12 (1H, m, Pyrrolidine-NCH₂), 2.61-2.36 (2H, m, CH₂N), 2.33 (3H, s, Ts-CH₃), 2.00-1.86 (1H, m, Pyrrolidine-NCH₂), 1.85-1.63 (3H, m, Pyrrolidine-CH₂), 1.63-1.49 (1H, m, Pyrrolidine-CH₂), 1.43 (5H, b.s, Boc-CH₃), 1.26 (4H, s, Boc-CH₃); ¹³C NMR (126 MHz, CDCl₃) δ 155.02, 142.99, 139.43, 138.30, 137.47, 129.26, 129.18, 128.51, 128.41, 128.02, 127.71, 127.56, 127.23, 79.38, 68.00, 63.24, 57.36, 50.06, 46.95, 29.30, 28.62, 23.96, 21.56; MS (ESI⁺) m/z 550.4 [M−H]⁺; HRMS calcd for C₃₁H₄₀N₃O₄S [M−H]⁺ 550.2734, found 550.2732 (0.3 ppm err)

### Example 20: Synthesis of Ligand 12

### tert-Butyl(S)-2-((((1S,2S)-2-((4-methylphenyl)sulfonamido)-1,2-diphenylethyl)amino)methyl)pyrrolidine-1-carboxylate

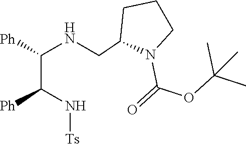 [SMILES: Cc1ccc(S(=O)(=O)N[C@@H](c2ccccc2)[C@@H](NC[C@@H]2CCCN2C(=O)OC(C)(C)C)c2ccccc2)cc1]

To a mixture of S,S-TsDPEN (183 mg, 0.5 mmol) and 4 Å molecular sieves in DCM (5 mL) was added dropwise a solution of (S)-Boc-prolinal (100 mg, 0.5 mmol) in DCM (2.5 mL). The molecular sieves were filtered off after stirring overnight and the solvent was removed under reduced pressure. The residue was dissolved in MeOH (5 mL) and acetic acid was added (6 drops) followed by slow addition of NaBH₃CN (38 mg, 0.6 mmol). After stirring overnight, the solvent was removed under reduced pressure and the residue was partitioned in EtOAc (10 mL) and H₂O (10 mL). The organic layer was collected and further extracted with EtOAc (2×10 mL). The organic extracts were combined, dried over MgSO₄, filtered and the solvent removed under reduced pressure. Purification by column chromatography (0-30% EtOAc in Pet. Ether) gave Ligand 12 as a white solid, which forms rotamers (173 mg, 0.32 mmol, 63%); ¹H NMR (500 MHz, CDCl₃) δ 7.42-7.29 (2H, m, ArH), 7.16-7.06 (3H, m, ArH), 7.05-6.98 (4H, m, ArH), 6.98-6.93 (2H, m, ArH), 6.93-6.86 (3H, m, ArH), 6.86-6.79 (1H, m, ArH), 4.36-4.15 (1H, m, CHPh), 4.05-3.73 (1H, m, CHPh), 3.72-3.51 (1H, m, Pyrrolidine-CH), 3.41-3.33 (1H, m, Pyrrolidine-NCH_{A}H_{B}), 3.26-3.18 (1H, m, Pyrrolidine-NCH_{A}H_{B}), 2.64-2.46 (1H, m, NHCH_{A}H_{B}), 2.42-2.34 (1H, m, NHCH_{A}H_{B}), 2.31 (3H, s, Ts-CH₃), 1.97-1.85 (1H, m, Pyrrolidine-CH₂), 1.84-1.70 (3H, m, Pyrrolidine-CH₂CH₂), 1.50 (6H, s, Boc-CH₃), 1.27 (3H, s, Boc-CH₃); ¹³C NMR (126 MHz, CDCl₃) δ 156.08, 142.50, 139.45, 138.59, 137.57, 129.18, 128.51, 128.21, 128.04, 127.83, 127.64, 127.46, 127.21, 79.70, 67.03, 63.71, 56.34, 51.63, 47.20, 29.89, 28.74, 24.11, 21.54; MS (ESI⁺) m/z 550.4 [M+H]⁺

### Example 21: Synthesis of Catalyst 9

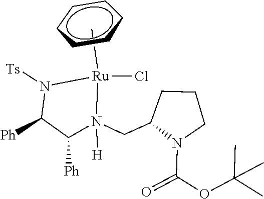

d.r. 5:1

To a Schlenk tube charged with benzene ruthenium (II) chloride dimer (50 mg, 0.1 mmol) and Ligand 11 (110 mg, 0.2 mmol) in PhCl (1.5 mL) was added triethylamine (81 mg, 0.8 mmol, 0.1 mL). After stirring at 80° C. for 1 h, the solution was allowed to cool to rt and the solvent removed under reduced pressure. The residue was dissolved in CHCl₃ (10 mL) and washed with water (10 mL). The organic layer was dried over MgSO₄, filtered and the solvent removed under reduced pressure to afford the crude mixture. Purification by column chromatography (10% EtOAc in DCM followed by 0-3% MeOH in DCM) afforded Catalyst 9 as a brown solid (136 mg, 0.18 mmol, 89%); vₘₐₓ 3482, 3192, 3060, 3027, 2971, 2924, 2875, 1681, 1600, 1494, 1478, 1454 cm⁻¹; ¹H NMR (500 MHz, CDCl₃) δ 7.33 (2H, d, J 8.1, ArH), 7.16-7.07 (3H, m, ArH), 7.06-6.98 (1H, m, ArH), 6.87-6.82 (3H, m, ArH), 6.80-6.73 (3H, m ArH), 6.59 (2H, d, J 7.4, ArH), 6.10 (6H, s, Benzene), 4.50-4.43 (1H, m, CHPh), 4.01 (1H, d, J 10.5, CHPh), 3.84 (1H, t, J 11.4, CH₂CHN), 3.70-3.63 (1H, m, CH_{A}H_{B}NBoc), 3.53 (1H, t, J 12.1, NH), 3.24-3.16 (1H, m, NCH_{A}H_{B}Pyr), 3.06-2.99 (1 H, m, NCH_{A}H_{B}Pyr), 2.32 (1H, t, J 11.4, CH_{A}H_{B}NBoc), 2.23 (3H, s, Ts-CH₃), 2.17-2.08 (1H, m, Pyrrolidine-CH₂), 1.67-1.62 (1H, m, Pyrrolidine-CH₂), 1.46-1.43 (1H, m, Pyrrolidine-CH₂), 1.42 (9H, s, (CH₃)₃, 1.25-1.11 (1H, m, Pyrrolidine-CH₂); ¹³C NMR (126 MHz, CDCl₃) δ 154.92, 141.31, 140.20, 139.62, 137.27, 128.82, 128.61, 128.50, 128.13, 128.01, 127.96, 127.24, 126.52, 84.68, 81.66, 79.74, 69.37, 55.79, 55.17, 46.95, 29.92, 28.56, 23.43, 21.37; MS (ESI⁺): m/z 728.3 [M−Cl]⁺; HRMS calcd for C₃₇H₄₄N₃O₄RuS [M−Cl]⁺ 728.2091, found 728.2104 (0.6 ppm error)

### Example 22: Synthesis of Catalyst 10

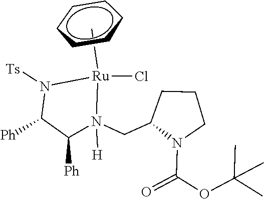

To a Schlenk tube charged with benzene ruthenium (II) chloride dimer (32 mg, 64 μmol) and Ligand 12 (70 mg, 0.13 mmol) in PhCl (1.2 mL) was added triethylamine (52 mg, 0.5 mmol, 70 μL). After stirring at 80° C. for 1 h, the solution was allowed to cool to rt and the solvent removed under reduced pressure. The residue was dissolved in CHCl₃ (10 mL) and washed with water (10 mL). The organic layer was dried over MgSO₄, filtered and the solvent removed under reduced pressure to afford the crude mixture. Purification by column chromatography (10% EtOAc in DCM followed by 0-3% MeOH in DCM) afforded Catalyst 10 as a brown solid (71 mg, 93 μmol, 73%); ¹H NMR (500 MHz, CDCl₃) δ 7.32 (2H, d, J 8.1, ArH), 7.09-7.01 (3H, m, ArH), 6.91 (1H, dd, J 10.4, 6.8, ArH), 6.84 (2H, d, J 6.8 Hz, ArH), 6.80 (2H, d, J 8.1, ArH), 6.67 (2H, d, J 7.6, ArH), 6.66-6.61 (1 H, m, ArH), 6.52 (2H, t, J 7.6, ArH), 6.03 (6H, s, Benzene), 4.42 (1H, d, J 10.8, NTsCHPh), 4.39-4.31 (1H, m, Pyrrolidine-CHNBoc), 3.87 (1H, ddd, J=13.1, 6.5, 2.5, NHCH_{A}H_{B}), 3.76 (1 H, t, J 10.8, NHCHPh), 3.05-2.96 (1H, m, Pyrrolidine-NBocCH_{A}H_{B}), 2.61 (1H, t, J 13.1, NHCH_{A}H_{B}), 2.22 (3H, s, Ts-CHs), 2.21-2.15 (1H, m, Pyrrolidine-NBocCH_{A}H_{B}), 1.95-1.85 (1 H, m, Pyrrolidine-CH₂), 1.62 (9H, s, (CH₃)₃), 1.60-1.53 (1H, m, Pyrrolidine-CH₂), 1.45-1.36 (1H, m, Pyrrolidine-CH₂), 1.26-1.15 (m, 1H, Pyrrolidine-CH₂); ¹³C NMR (126 MHz, CDCl₃) δ 157.84, 143.86, 139.13, 138.98, 137.48, 129.96, 128.90, 128.19, 128.11, 127.88, 126.68, 126.41, 125.79, 83.84, 81.06, 76.13, 71.42, 61.15, 56.55, 46.29, 29.75, 28.78, 23.83, 21.35; MS (ESI⁺) m/z 728.3 [M−Cl]⁺; HRMS calcd for C₃₇H₄₄N₃O₄RuS [M−Cl]⁺ 728.2091, found 728.2094 (0.8 ppm error)

### Use of Organometallic Catalysts of Examples 7 to 11, 16 to 18 in an Asymmetric Transfer Hydrogenation

The organometallic catalysts of Examples 7 to 11, and Comparative Examples 1 and 2, were used in asymmetric transfer hydrogenation reactions of ketones.

### Comparative Example 1: Ligand CE1

The ligand for use with an organometallic catalyst of Comparative Example 1 CE1 has the following formula:

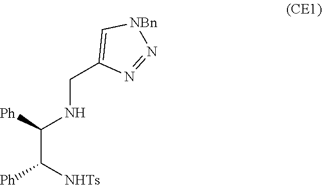

CE1 was synthesised according to the procedure described in Johnson, T. C.; Tatty, W. G.; Wills, M. Organic Letters, 2012, 14, 5230-5233,

### Comparative Example 2: Ligand CE2

The ligand for use with an organometallic catalyst of Comparative Example 2 CE2 has the following formula:

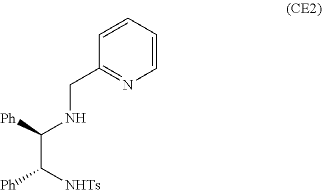

CE2 was synthesised according to the procedure described in Moftah O. Darwish, Alistair Wallace, Guy J. Clarkson and Martin Wills, Tetrahedron Lett. 2013, 54, 4250-4253.

### Comparative Example 3: Synthesis of Ligand CE3

### N-((1R,2R)-1,2-Diphenyl-2-((((S)-pyrrolidin-2-yl)methyl)amino)ethyl)-4-methylbenzenesulfonamide

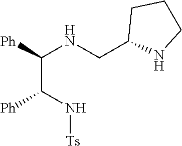 [SMILES: Cc1ccc(S(=O)(=O)N[C@H](c2ccccc2)[C@H](NC[C@@H]2CCCN2)c2ccccc2)cc1]

To a solution of Ligand 11 (100 mg, 0.18 mmol) in DCM (2 mL) was added dropwise trifluoroacetic acid (0.14 mL). After stirring overnight, the mixture was concentrated under reduced pressure and the residue partitioned in DCM (10 mL) and sat. NaHCO₃(10 mL). Following extraction with DCM (3×10 mL) the organic extracts were combined, dried over MgSO₄, filtered and the solvent was removed under reduced pressure. Purification by column chromatography (50% EtOAc in Pet. Ether) gave Ligand CE3 as a white solid (80 mg, 0.18 mmol, 98%); [α]_{D}+27.5 (c 0.1 in CHCl₃); ¹H NMR (500 MHz, CDCl₃) δ 7.40 (2H, d, J 8.2, ArH), 7.13-7.07 (3H, m, ArH), 7.04-7.00 (3H, m, ArH), 6.97 (2H, t, J 7.2, ArH), 6.94-6.90 (2H, m, ArH), 6.88 (2H, d, J 7.2, ArH), 4.32 (1H, d, J 8.5, NHCHPh), 3.65 (1H, d, J 8.5, NTsCHPh), 3.28-3.19 (1H, m, Pyrrolidine-CH), 3.03-2.91 (2H, m, Pyrrolidine-NHCH₂), 2.47 (1H, dd, J 11.8, 4.5, NHCH_{A}H_{B}), 2.32 (3H, s, Ts-CH₃), 2.28 (1H, dd, J 11.8, 8.4, NHCH_{A}H_{B}), 1.86-1.67 (3H, m, Pyrrolidine-CH₂CH₂), 1.34-1.23 (1H, m, Pyrrolidine-CH₂). ¹³C NMR (126 MHz, CDCl₃) δ 142.69, 139.68, 138.49, 137.53, 129.18, 128.37, 127.90, 127.84, 127.68, 127.49, 127.23, 68.45, 63.48, 58.65, 52.12, 46.39, 29.43, 25.41, 21.55; MS (ESI⁺) m/z 450.3 [M+H]⁺; HRMS calcd for C₂₆H₃₂N₃O₂S [M+H]⁺450.2210, found 450.2213 (0.8 ppm error)

### Comparative Example 4: Synthesis of Ligand CE4

### S,S-L2 (JB461)-N-((1S,2S)-1,2-Diphenyl-2-((((S)-pyrrolidin-2-yl)methyl)amino)ethyl)-4-methylbenzenesulfonamide

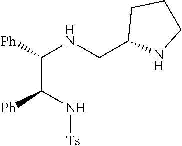 [SMILES: Cc1ccc(S(=O)(=O)N[C@@H](c2ccccc2)[C@@H](NC[C@@H]2CCCN2)c2ccccc2)cc1]

To a solution of Ligand 12 (45 mg, 82 μmol) in DCM (1.5 mL) was added dropwise trifluoroacetic acid (94 mg, 820 μmol). After stirring overnight, the mixture was concentrated under reduced pressure and the residue partitioned in DCM (10 mL) and sat. NaHCO₃(10 mL). Following extraction with DCM (3×10 mL) the organic extracts were combined, dried over MgSO₄, filtered and the solvent was removed under reduced pressure. Purification by column chromatography (50% EtOAc in Pet. Ether) gave Ligand CE4 as a white solid (36 mg, 80 μmol, 98%); [α]_{D}+28.1 (c 0.1 in CHCl₃); ¹H NMR (500 MHz, CDCl₃) δ 7.37 (2H, d, J 8.2, ArH), 7.14-7.07 (3H, m, ArH), 7.00 (3H, d, J 8.2, ArH), 6.96 (2H, t, J 7.5, ArH), 6.94-6.90 (2H, m, ArH), 6.88 (2H, d, J 7.5, ArH), 4.36 (1H, d, J 8.5, NTsCHPh), 3.65 (1H, d, J 8.5, NHCHPh), 3.25 (1H, b.s, Pyrrolidine-CH), 3.04-2.90 (2H, m, Pyrrolidine-NHCH₂), 2.47 (1H, dd, J 11.8, 4.5, NHCH_{A}H_{B}), 2.32 (3H, s, Ts-CH₃), 2.31-2.25 (1H, m, NHCH_{A}H_{B}), 1.86-1.67 (3H, m, Pyrrolidine-CH₂CH₂), 1.36-1.28 (1H, m, Pyrrolidine-CH₂CH₂); ¹³C NMR (126 MHz, CDCl₃) δ 142.62, 139.72, 138.39, 137.67, 129.12, 128.37, 127.88, 127.68, 127.48, 127.20, 68.41, 63.41, 58.74, 52.03, 46.44, 29.39, 25.39, 21.54; MS (ESI⁺) m/z 450.3 [M+H]⁺; HRMS calcd for C₂₆H₃₂N₃O₂S [M+H]⁺ 450.2210, found 450.2205 (1.1 ppm error)

### Comparative Example 5: Synthesis of Ligand CE5

### N-((1R,2R)-2-((((S)-1-Methylpyrrolidin-2-yl)methyl)amino)-1,2-diphenylethyl)-4-methylbenzenesulfonamide

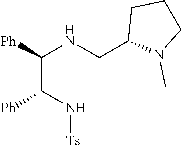 [SMILES: Cc1ccc(S(=O)(=O)N[C@H](c2ccccc2)[C@H](NC[C@@H]2CCCN2C)c2ccccc2)cc1]

A solution of Ligand 11 (100 mg, 0.18 mmol) in THF (4 mL) was cooled to 0° C. and was added LiAlH₄ (2M in hexanes, 0.18 mL, 0.36 mmol). After refluxing for 3 hours to mixture was allowed to cool and the reaction was quenched with EtOAc. Rochelle salt solution (10 mL) was added and stirred for 30 mins. Following extraction with EtOAc (3×10 mL) the organic extracts were combined, dried over MgSO₄, filtered and the solvent removed under reduced pressure. Purification by column chromatography (0-10% MeOH in DCM) gave Ligand CE5 as a white solid (23 mg, 50 μmol, 28%); ¹H NMR (500 MHz, MeOD) δ 7.38 (2H, d, J 8.2, ArH), 7.14-7.07 (5H, m, ArH), 7.03 (2H, d, J 8.2, Ar), 6.95-6.90 (1H, m, J 7.2, ArH), 6.87 (2H, t, J 7.4, ArH), 6.74 (2H, d, J 7.4, ArH), 4.42 (1H, d, J 9.7, NTsCHPh), 3.82 (1H, d, J 9.7, NHCHPh), 3.68-3.60 (1H, m, NMeCH_{A}H_{B}), 3.29-3.21 (1H, m, CHNMe), 3.10-3.01 (1H, m, NMeCH_{A}H_{B}), 2.80 (1H, dd, J 13.4, 6.4, NHCH_{A}H_{B}), 2.72 (3H, s, NCH₃), 2.66 (1H, dd, J 13.4, 5.0, NHCH_{A}H_{B}), 2.27 (3H, s, TsCH₃), 2.24-2.17 (1H, m, Pyrrolidine-CH₂), 2.14-1.96 (2H, m, Pyrrolidine-CH₂), 1.82-1.72 (1H, m, Pyrrolidine-CH₂); ¹³C NMR (126 MHz, MeOD) δ 144.22, 140.93, 139.37, 139.33, 130.20, 129.45, 129.37, 128.83, 128.80, 128.72, 128.05, 127.94, 69.67, 69.37, 65.26, 57.60, 47.76, 41.27, 28.60, 23.12, 21.31; MS (ESI⁺) m/z 464.3 [M+H]⁺; HRMS calcd for C₂₇H₃₄N₃O₂S [M+H]⁺464.2366, found 464.2369 (0.5 ppm error)

The following General Procedures 1 to 3 may be followed for asymmetric transfer hydrogenation reactions of ketones using the ligands and/or catalysts according to the invention. General Procedure 4 was followed to produce racemic alcohols for comparative analysis.

### General Procedure 1: Ru(Arene)/Ligand Reduction

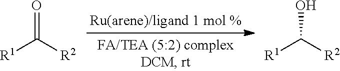

Catalyst, e.g. of Examples 7 to 11, (1 mol %) in FA/TEA 5:2 complex (0.5 mL) was stirred under an inert atmosphere at rt for 15 min. The ketone (1 mmol) in dichloromethane (0.5 mL) was then added to the mixture and stirred at rt until completion. Sat. NaHCO₃(10 mL) was added and the product extracted with EtOAc (10 mL). The aqueous layer was further extracted with EtOAc (2×10 mL). Organic fractions were then combined, dried over MgSO₄, filtered and the solvent removed under pressure. The crude products were purified by column chromatography (0-50% EtOAc in Pet. Ether). Enantiomeric excess was determined by chiral GC or HPLC.

### General Procedure 2: In Situ Procedure Using the Ligand and [RuCl₂(benzene)]₂ Separately

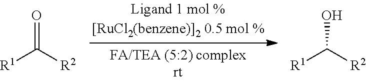

To a Schlenk tube charged with [RuCl₂(Benzene)]₂ (2.50 mg, 0.5 mmol) and ligand (1 mmol) was added formic acid/triethylamine 5:2 complex (0.5 mL). After stirring for 30 min, the ketone (1 mmol) was added and left to stir. Upon completion, NaHCO₃(10 ml) was added and the product was extracted with EtOAC (3×10 mL). The organic extracts were combined, dried over MgSO₄, filtered and the solvent removed under reduced pressure. The crude products were purified by column chromatography (0-50% EtOAc in Pet. Ether). Enantiomeric excess was determined by chiral GC or HPLC.

### General Procedure 3: In Situ Procedure Using the Ligand and Ru₃(CO)₁₂ Separately

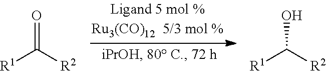

A mixture of ligand (5 mol %) and Ru₃(CO)₁₂ (5/3 mol %) in iPrOH (10 mL) was stirred at 80° C. under an inert atmosphere for 30 min in a Schlenk tube. Ketone (1 mmol) was then added to the solution and the resulting mixture was stirred at 80° C. for 72 h. The solvent was removed under reduced pressure and the crude product was purified by column chromatography (0-50% EtOAc in Pet. Ether). Enantiomeric excess was determined by GC or chiral HPLC.

### General Procedure 4: Racemic Alcohols Procedure for Comparative Analysis

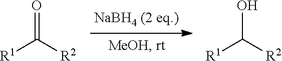

To a solution of ketone (1 eq.) in MeOH (0.1 M) was added NaBH₄ (2 eq.) portion-wise. The solution was stirred at rt until the ketone had consumed. The solvent was then removed under reduced pressure and the residue partitioned between water and EtOAc. The organic extract was collected and the aqueous layer extracted a further 2 times with EtOAc. Organic layers were combined, dried over MgSO₄, filtered and the solvent removed under reduced pressure. Products were purified by column chromatography gradient elution 0-40% EtOAc in Pet. ether.

### General Procedure 5: Reduction of Cyclic Imines

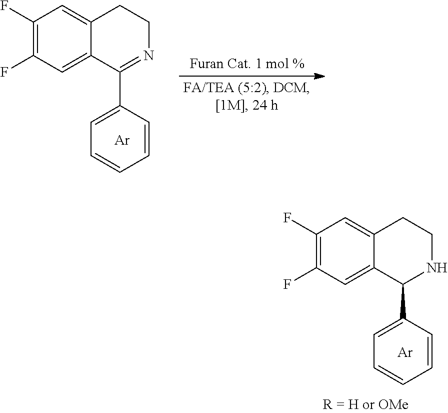

To a Schlenk tube charged with the catalyst (5 μmol) was added formic acid/triethylamine 5:2 (0.25 mL) and left to stir for 15 min under a N₂ atmosphere. A solution of the imine (0.5 mmol) in DCM (0.25 mL) was added to the mixture and left to stir at rt overnight. The reaction was quenched with sat. NaHCO₃ (5 mL) and extracted with EtOAc (3×5 mL). The organic layers were combined, dried over MgSO₄, filtered and the solvent removed under reduced pressure. The product was purified by column chromatography gradient elution 0-60% EtOAc in Pet. Ether.

Referring first to Table 1, there is shown the results for asymmetric transfer hydrogenations using General Procedure 1 for the reduction of ketones using Catalysts 1 to 4 according to the invention.

Referring also to Table 2, there is shown the results for asymmetric transfer hydrogenations using General Procedure 2 for the reduction of ketones using Ligands 1 to 4 to form Catalysts in situ according to the invention.

Referring also to Table 3, there is shown the results for asymmetric transfer hydrogenations using General Procedure 5 for the reduction of imines using Catalyst 1 according to the invention.

TABLE 1
Asymmetric Transfer Hydrogenations using General Procedure 1 for the reduction of ketones using Catalysts according to the invention

| | | Example or Comparative Example | | | | | | |
|---|---|---|---|---|---|---|---|---|
| | | No | Ligand of | Ligand of | Catalyst | Catalyst | Catalyst | Catalyst |
| | | Chiral | Comparative | Comparative | 1 of | 2 of | 3 of | 4 of |
| | | Catalyst | Example CE1 | Example CE2 | Example 7 | Example 8 | Example 9 | Example 10 |
| | | General Procedure | | | | | | |
| | | 4 | 3 | 3 | 1 | 1 | 1 | 1 |
| | Substrate | Reaction time/Yield and/or Conversion/Enantiomeric Excess (ee) | | | | | | |
| 1ⁱ | 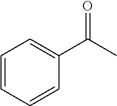 | Racemic | 48 hours Yield: 98% 92% ee | 48 hours Yield: 80% 94% ee | ^{a}72 hours (conv. 95%) 90% ee | ^{b}72 hours (conv. 56%) 93% ee | ^{c}72 hours (conv. 99%) 95% ee | ^{d}95 hours (conv. 63%) 95% ee |
| 2 | 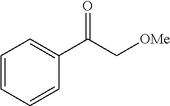 [SMILES: COCC(=O)c1ccccc1] | Racemic | No reaction | No reaction | 5 days Yield: 84% 94% ee | 5 days Yield: 41% 91% ee | 6 days Yield: 74% 92% ee | 7 days Yield: 79% (82% conv.) 91% ee |
| 3 | 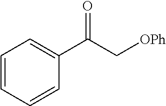 [SMILES: O=C(COc1ccccc1)c1ccccc1] | Racemic | 72 hours Yield: 94% (Full conv.) 81% ee | 72 hours Yield: 96% (Full conv.) 53% ee | 72 hours Yield: 71% (Full conv.) 92% ee | 120 hours Yield: 89% (98% conv.) 94% ee | 96 hours Yield: 91% (Full conv.) 90% ee | 6 days Yield: 97% (Full conv.) 92% ee |
| 4 | 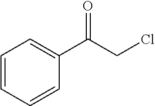 | Racemic | No reaction | No reaction | 96 hours Yield: 91% (99% conv.) 90% ee | 96 hours Yield: 79% (Full conv.) 89% ee | 96 hours Yield: 87% (99% conv.) 91% ee | 7 days Yield: 86% (94% conv.) 91% ee |
| 5 | 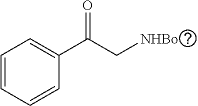 | Racemic | 72 hours Yield: 99% (Full conv.) 97% ee | 72 hours Yield: 92% (Full conv.) 94% ee | 120 hours Yield: 94% (Full conv.) 97% ee | 120 hours Yield: 95% (Full conv.) 96% ee | 120 hours Yield: 95% (Full conv.) 93% ee | 7 days Yield: 91% (98% conv.) 95% ee |
| 6 | 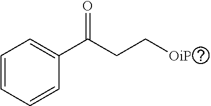 | Racemic | 72 hours Yield: 95% (Full conv.) 99% ee | 72 hours Yield: 96% (Full conv.) 99% ee | 120 hours Yield: 70% (80% conv.) 89% ee | 132 hours Yield: 45% (52% conv.) 92% ee | 120 hours Yield: 74% (87% conv.) 94% ee | 7 days Yield: 41% (55% conv.) 96% ee |
| 7 | 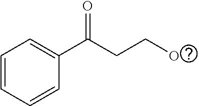 [SMILES: O=C(CCO)c1ccccc1] | Racemic | 72 hours Yield: 98% (Full conv.) 86% ee | 72 hours Yield: 96% (Full conv.) 90% ee | 120 hours Yield: 94% (98% conv.) 91% ee | 120 hours Yield: 41% (50% conv.) 87% ee | 6 days Yield: 93% (Full conv.) 91% ee | 7 days Yield: 70% (72% conv.) 90% ee |
| 8 | 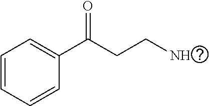 [SMILES: NCCC(=O)c1ccccc1] | Racemic | 72 hours Yield: 95% 92% ee | 72 hours Yield: 88% 91% ee | 120 hours Yield: 88% (95% conv.) 91% ee | 120 hours Yield: 41% (52% conv.) 91% ee | 144 h Yield: 84% (85% conv.) 92% ee | 6 days Yield: 92% (96% conv.) 96% ee |
| 9 | 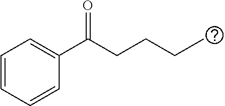 | Racemic | 72 hours Yield: 91% (99% conv.) 83% ee | 72 hours Yield: 89% (95% conv.) 89% ee | 144 hours Yield: 70% (89% conv.) 88% ee | 144 hours Yield: 49% (55% conv.) 84% ee | 168 hours Yield: 57% (66% conv.) 90% ee | 7 days Yield: 66% (71% conv.) 92% ee |
| 10 | 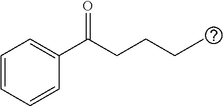 | Racemic | 72 hours Yield: 98% (Full conv.) 90% ee | 72 hours Yield: 98% (Full conv.) 90% ee | 72 hours Yield: 93% (93% conv.) 90% ee | 7 days Yield: 61% 87% ee | 6 days Yield: 96% (Full conv.) 89% ee | 7 days Yield: 48% (50% conv.) 93% ee |
| 11 | 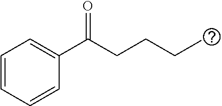 | Racemic | 72 hours Yield: 99% (Full conv.) 92% ee | 72 hours Yield: 92% (Full conv.) 91% ee | ^{e}80 hours Yield: 92% (Full conv.) 93% ee | ^{f}144 hours Yield: 45% (55% conv.) 88% ee | ^{g}144 hours Yield: 73% (77% conv.) 76% ee | ^{h}7 days Yield: 45% (59% conv.) 90% ee |
| 12 | 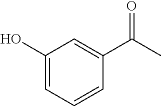 | Racemic | 48 hours (98% conv.) 94% ee | 48 hours (99% conv.) 88% ee | 96 hours Yield: 81% (Full conv.) 93% ee | 144 hours Yield: 74% (94% conv.) 92% ee | 144 hours Yield: 86% (94% conv.) 93% ee | 4 days Yield: 87% (99% conv.) 90% ee |
| 13 | 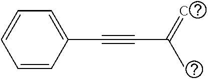 | No reduction | No reduction | No reduction | 4 days Yield: 80% 86% ee | 5 days Yield: 57% 89% ee | 5 days Yield: 65% 86% ee | 5 days Yield: 45% (56% conv.) 84% ee |

^{a}Using General Procedure 1 with Catalyst 1 of Example 7 using a 2M concentration of Substrate 1, the following results were obtained: 24 hours, 99% conversion, 92% ee.
^{b}Using General Procedure 1 with Catalyst 2 of Example 8 using a 2M concentration of Substrate 1, the following results were obtained: 24 hours, 98% conversion, 92% ee.
^{c}Using General Procedure 1 with Catalyst 3 of Example 9 using a 2M concentration of Substrate 1, the following results were obtained: 72 hours, 96% conversion, 90% ee.
^{d}Using General Procedure 1 with Catalyst 4 of Example 10 using a 2M concentration of Substrate 1, the following results were obtained: 6.5 days, 90% conversion, 93% ee.
^{e}Using General Procedure 2 with Ligand 1 using a 2M concentration of Substrate 11, the following results were obtained: 7 days, 96% conversion, 88% ee.
^{f}Using General Procedure 2 with Ligand 2 using a 2M concentration of Substrate 11, the following results were obtained: 7 days, 47% conversion, 83% ee.
^{g}Using General Procedure 2 with Ligand 3 using a 2M concentration of Substrate 11, the following results were obtained: 7 days, 41% conversion, 90% ee.
^{h}Using General Procedure 2 with Ligand 2 using a 2M concentration of Substrate 11, the following results were obtained: 7 days, 41% conversion, 89% ee.
ⁱUsing General Procedure 1 with Catalyst 5 of Example 11, the following results were obtained: 86 h, 73% conversion, 95% ee.

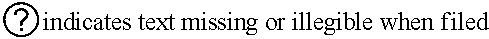

TABLE 1a
Alcohol products obtained from asymmetric reduction of the ketone
precursor using Catalyst 1 of Example 7 in General Procedure 1

| Alcohol Product | Yield | Enantioselectivity |
|---|---|---|
| 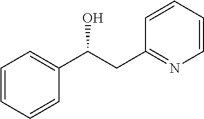 [SMILES: O[C@H](Cc1ccccn1)c1ccccc1] | 90% | 98% ee |
| 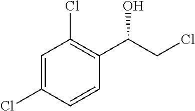 [SMILES: O[C@H](CCl)c1ccc(Cl)cc1Cl] | 97% | 53% ee |

TABLE 2
Asymmetric Transfer Hydrogenation using General Procedure 2 for the reduction of ketones
using Ligands to form Catalysts in situ according to the invention

| | | Example or Comparative Example | | | | |
|---|---|---|---|---|---|---|
| | | | Catalyst 1 | Catalyst 2 | Catalyst 3 | Catalyst 4 |
| | | No Chiral | of Example | of Example | of Example | of Example |
| | | Catalyst | 7 | 8 | 9 | 10 |
| | | General Procedure | | | | |
| | | 4 | 2 | 2 | 2 | 2 |
| | Substrate | Reaction time/Yield and/or Conversion/Enantiomeric Excess (ee) | | | | |
| 14 | 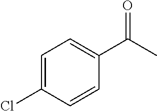 | Racemic | 47 hours (conv. 99.8%) 87% ee | 94 hours (conv. 99%) 87% ee | 156 hours (conv. 99%) 89% ee | 96 hours (conv. 99%) 90% ee |
| 15 | 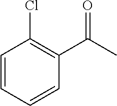 | Racemic | 72 hours (conv. 99%), 57% ee | 165 hours (conv. 99%) 57% ee | 186 hours (conv. 80%) 66% ee | 96 hours (conv. 98%) 61% ee |
| 16^{j} | 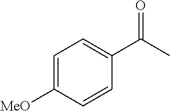 | Racemic | 184 hours (conv. 91%) 87% ee | 200 hours (conv. 70%) 87% ee | 190 hours (conv. 63%) 87% ee | 96 hours (conv. 51%) 87% ee |
| 17^{k} | 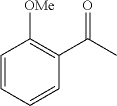 | Racemic | 164 hours (conv. 90%) 65% ee | 166 hours (conv. 87%) 66% ee | 166 hours (conv. 63%) 72% ee | 164 hours (conv. 97%) 68% ee |
| 18 | 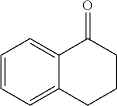 [SMILES: O=C1CCCc2ccccc21] | Racemic | 72 hours (conv. 99.6%) 99% ee | 96 hours (conv. 95%) 99% ee | 96 hours (conv. 96%) 99% ee | 70 hours (conv. 99%) 99% ee |
| 19 | 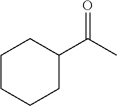 | Racemic | 144 hours (conv. 92%) 53% ee | 144 hours (conv. 75%) 35% ee | 144 hours (conv. 70%) 36% ee | 120 hours (conv. 98%) 48% ee |
| 20 | 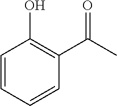 | Racemic | 96 hours (conv. 97%) 94% ee | 168 h (conv. 99%) 93% ee | 168 h (conv. 99%) 95% ee | 72 h (conv. 99%) 96% ee |

^{j}Using General Procedure 1 with Catalyst 4 of Example 10, the following results were obtained: 72 hours, 47% conversion, 85% ee.
^{k}Using General Procedure 1 with Catalyst 1 of Example 7, the following results were obtained: 72 hours, 98% conversion, 64% ee; Using General Procedure 1 with Catalyst 3 of Example 9, the following results were obtained: 72 hours, 97% conversion, 72% ee.

TABLE 3
Asymmetric Transfer Hydrogenation using General Procedure 5 for the
reduction of imines using Catalyst 1 according to the invention

| | | Reaction Time; |
|---|---|---|
| | | Yield and/or |
| | | Conversion; |
| | | Enantiomeric |
| | Amine Product of Reaction | Excess (ee) |
| 1 | 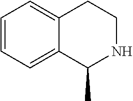 | 95% conv. 80% ee |
| 2 | 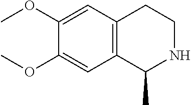 | 96 h 96% conv. 81% ee |
| 3^{m} | 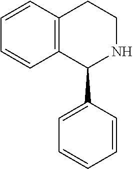 [SMILES: c1ccc([C@@H]2NCCc3ccccc32)cc1] | 96 h 71% yield, 98% conv. 90% ee |
| 4 | 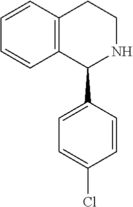 [SMILES: Clc1ccc([C@@H]2NCCc3ccccc32)cc1] | 64% yield 88% ee |
| 5 | 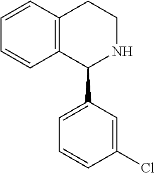 [SMILES: Clc1cccc([C@@H]2NCCc3ccccc32)c1] | 62% yield 91% ee |
| 6 | 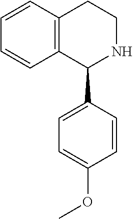 [SMILES: COc1ccc([C@@H]2NCCc3ccccc32)cc1] | 87% yield 92% ee |
| 7 | 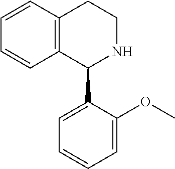 [SMILES: COc1ccccc1[C@@H]1NCCc2ccccc21] | 28% yield 90% ee |
| 8 | 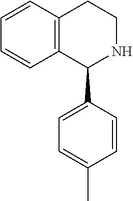 [SMILES: Cc1ccc([C@@H]2NCCc3ccccc32)cc1] | 74% yield 90% ee |
| 9 | 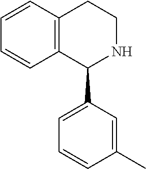 [SMILES: Cc1cccc([C@@H]2NCCc3ccccc32)c1] | 76% yield 92% ee |
| 10 | 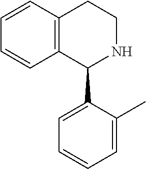 [SMILES: Cc1ccccc1[C@@H]1NCCc2ccccc21] | 25% yield 76% ee |
| 11 | 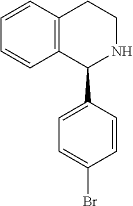 [SMILES: Brc1ccc([C@@H]2NCCc3ccccc32)cc1] | 79% yield 93% ee |
| 12 | 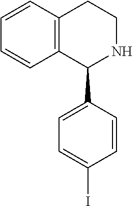 [SMILES: Ic1ccc([C@@H]2NCCc3ccccc32)cc1] | 57% yield 93% ee |
| 13 | 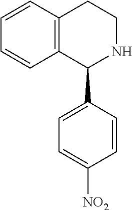 [SMILES: O=[N+]([O-])c1ccc([C@@H]2NCCc3ccccc32)cc1] | 50% yield 89% ee |
| 14 | 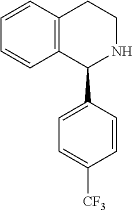 [SMILES: FC(F)(F)c1ccc([C@@H]2NCCc3ccccc32)cc1] | 90% yield 91% ee |
| 15 | 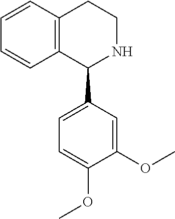 [SMILES: COc1ccc([C@@H]2NCCc3ccccc32)cc1OC] | 81% yield 91% ee |
| 16 | 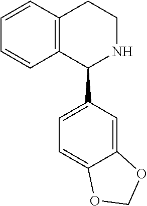 [SMILES: c1ccc2c(c1)CCN[C@H]2c1ccc2c(c1)OCO2] | 72% yield 90% ee |
| 17 | 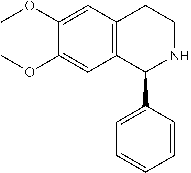 [SMILES: COc1cc2c(cc1OC)[C@H](c1ccccc1)NCC2] | 72% yield 92% ee |
| 18 | 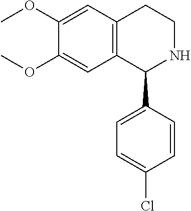 [SMILES: COc1cc2c(cc1OC)[C@H](c1ccc(Cl)cc1)NCC2] | 82% yield 97% ee |
| 19 | 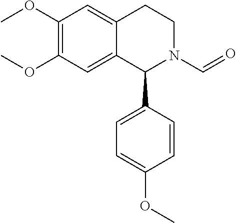 [SMILES: COc1ccc([C@H]2c3cc(OC)c(OC)cc3CCN2C=O)cc1] | 26% yield (for formylated)ⁿ 95% ee |
| 20 | 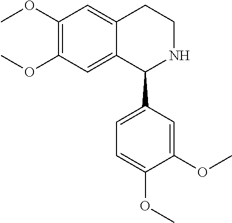 | 77% yield 93% ee |

^{m}(i) Using Catalyst 1 in General Procedure 5 using MeCN in place of DCM, the enantiomeric excess was 90% ee; (ii) using Catalyst 2 in General Procedure 5, the following results were obtained: 48 hours, 86% conversion, 91% ee; (iii) Using Catalyst 4 in General Procedure 5, the following results were obtained: 96 hours, 77% conversion, 49% ee; (iv) Using a catalyst according to a comparative example wherein the catalyst has the structure of Catalyst 1 comprising a phenyl ring in place of the furan provided the following results: 12% conversion, 0% ee.
ⁿThe imine was fully reduced by TLC. However, a mixture of products was formed (formylated and non-formylated) with the formyl-product as the major product, which was isolated.

It is shown that the enantioselectivity are improved over prior art catalysts for analogous imine reductions (for example, those described in Marc Perez, Zi Wu, Michelangelo Scalone, Tahar Ayad, and Virginie Ratovelomanana-Vidal, Eur. J. Org. Chem. 2015, 6503-6514). Product 3, result in paper (12a) is 29% ee, Product 6, result in paper (12l) is 39% ee, product 7, result in paper (12f) is 79% ee, product 8, result in paper (12k) is 36% ee, product 9, result in paper (12i) is 36% ee, product 17, result in paper (11i) is 82% ee, our product 20, result in paper (11j) is 75% ee.

Table 4a, 4b, 4c Asymmetric Transfer Hydrogenation for the reduction of acetophenone

TABLE 4a
Reduction of acetophenone using General Procedure 3

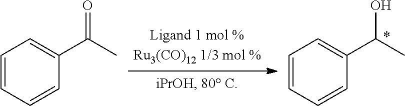

| Ligand | Time | Conversion | ee | R/S |
|---|---|---|---|---|
| Ligand 11 | 96 | 67 | 27 | S |
| Ligand 7 | 96 | 6 | 0 | — |
| CE3 | 48 | 85 | 0 | — |
| CE5 | 96 | 35 | 5 | R |
| CE4 | 48 | 45 | 0 | — |

The invention aims to improve the enantioselectivity of asymmetric hydrogenation reactions by redesigning the ligands and metal complexes used as catalysts. It would be advantageous to be able to add additional functionality to the X group of the ligand with the aim of improving enantioselectivity. However, it has previously been shown that tridentate ligands wherein X comprises a nitrogen atom that is able to function as a third donor group form inactive catalysts because the third donor group coordinates to the metal centre and inhibits catalysis. It has been shown that these ligands are able to form catalytically active complexes with Ru₃(CO)₁₂. However, it would be advantageous to provide ligands with a third donor group that are able to form active catalysts in a TsDPEN/Rutarene/Cl-type complex with the aim of improving the enantioselectivity of an asymmetric hydrogenation reaction.

It is shown from the results in Table 4a that the nature of the heterocyclic group can influence the effectiveness of the catalysts, such that ligands containing strong N-donor groups as the third ligand are suited to catalysis using Ru₃(CO)₁₂ as the metal source whereas those with weak donors such as heterocycles or esters containing oxygen at the position adjacent to the TsDPEN form effective complexes of the [TsDPEN/Rutarene/Cl] type, as shown from the results of Tables 4b and 4c.

TABLE 4b
Reduction of acetophenone using General Procedure 2^{p}

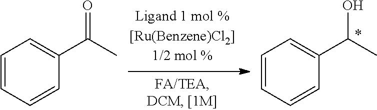

| Ligand | Time | Conversion | ee | R/S |
|---|---|---|---|---|
| CE3 | — | No reduction | — | — |
| CE5 | — | No reduction | — | — |
| CE4 | — | No reduction | — | — |
| Ligand 8 | 96 | 42 | 77 | R |
| Ligand 9 | 240 | 97 | 85 | R |
| Ligand 10 | 24 h | 27 | 90 | R |

^{p}(i) Using Ligand 9 with [Ru(benzene)Cl₂]₂ in water/sodium formate gave the following results: 120 h, 30% cony. 78% ee; (ii) Using Ligand 9 with [Ru(C₆H₅CH₂OEt)Cl₂]₂−FA/TEA, DCM, [1 M] gave the following results: 264 h, 82% cony, 85% ee.

TABLE 4c
Reduction of acetophenone using General Procedure 1

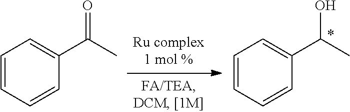

| Catalyst | Time | Conversion | Ee | R/S |
|---|---|---|---|---|
| Catalyst 9 of | 72 h | 83 | 92 | R |
| Example 21 | | | | |
| Catalyst 6 of | 116 h | 34 | 89 | R |
| Example 16 | | | | |
| Catalyst 10 of | — | No reduction | — | — |
| Example 22 | | | | |
| Catalyst 7 of | 72 | 94 | 91 | R |
| Example 17 | | | | |
| Catalyst 8 of | 66 h | 36 | 92 | R |
| Example 18 | | | | |

It has been surprisingly found that ligands according to the invention, which would be expected to bind in a tridentate manner, may be used to form organometallic complexes for use as a “single reagent” catalyst in asymmetric synthesis, for example, in asymmetric transfer hydrogenations.

This is surprising since the prior art teaches that tridentate ligands form inactive complexes with metal centres, which inhibits catalytic activity.

The ligands according to the invention are advantageous because these combine the electron donating effects of tridentate ligands, with the ability to form a “single reagent” [η⁶-arene)Ru(II)TsDPEN(Cl)] catalyst, to produce reduced substrates in high enantiomeric excess and high conversion.

The high yield and enantiomeric excess of chiral alcohols produced in asymmetric transfer hydrogenations using Catalysts 7 to 11 according to the invention has been demonstrated, the results of which are shown in Table 1 and Table 2 above. The results using Substrates 2, 4, and 13 are particularly striking, in that no reduction reaction was observed using Catalysts CE1 and CE2, but each of Catalysts 7 to 11 according to the invention produced chiral alcohol products in both moderate to high yield and enantiomeric excess (ee).

It will be appreciated by those skilled in the art that several variations to the aforementioned embodiments are envisaged without departing from the scope of the invention.

It will also be appreciated by those skilled in the art that any number of combinations of the aforementioned features and/or those shown in the appended drawings provide clear advantages over the prior art and are therefore within the scope of the invention described herein.

1. A compound, e.g. a diamine ligand, represented by the following general formula (1): 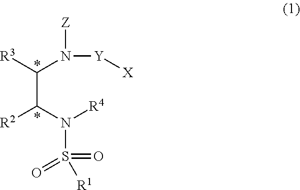
wherein
each * represents an asymmetric carbon atom;
X represents a group selected from one of an ester (e.g. a t-butyl ester); a thioester; an amide; a heterocyclic moiety (e.g. a five-membered heterocyclic ring) comprising one or more of O, S, Se, and/or P (e.g. a furan, a tetrahydrofuran, a thiophene, an isoxazole, a bromo-furan, or a thiazole); a moiety (e.g. a five-membered heterocyclic ring) comprising a nitrogen atom, wherein the nitrogen atom is protected with a protecting group containing an electron-withdrawing group, preferably the protecting group is selected from one of a carbamate protecting group, an amide protecting group, an aryl sulphonamide protecting group, or an alkyl sulphonamide protecting group; and optionally X may additionally comprise a solid support, e.g. a polymeric or a silica particle;
Y represents or is CtT′T″ where ‘t’ is 0 or 1 and when T is 1 T′ and T″ may individually represent a substituent, e.g. if t is 1, T′ and/or T″ may each be hydrogen or deuterium atom, or a halogen atom; for example, Y may represent a carbon atom comprising two further substituents, e.g. Y represents CH₂;
Z represents a hydrogen atom or a deuterium atom;
R¹ represents an alkyl group (e.g. a functionalised alkyl group) preferably having between 1 to 100 carbon atoms, for example, between 1 to 30 carbon atoms (e.g. 1 to 20 carbon atoms, or 1 to 10 carbon atoms), a halogenated alkyl group preferably having between 1 to 100 carbon atoms (e.g. CF₃), for example, between 1 to 30 carbon atoms (e.g. 1 to 20 carbon atoms, or 1 to 10 carbon atoms), an aryl group preferably having between 5 to 100 carbon atoms, e.g. 6 to 30 carbon atoms and optionally having one or more substituents selected from alkyl groups preferably having 1 to 100 carbon atoms, e.g. 1 to 10 carbon atoms, halogenated alkyl groups preferably having 1 to 100 carbon atoms, e.g. 1 to 10 carbon atoms, and/or halogen atoms; or R¹ represents a solid support, e.g. a silica particle or a polymeric particle;
R² and R³ each independently represent a group selected from alkyl groups preferably having between 1 to 100 carbon atoms, for example 1 to 20 carbon atoms (e.g. 1 to 10 carbon atoms), aryl groups (e.g. phenyl groups), and cycloalkyl groups preferably having 3 to 8 carbon atoms, the aryl group or phenyl group optionally having one or more substituents selected from alkyl groups preferably having between 1 to 100 carbon atoms, e.g. between 1 to 20 carbon atoms (e.g. 1 to 10 carbon atoms), alkoxy groups preferably having between 1 to 100 carbon atoms, for example, between 1 to 20 carbon atoms (e.g. 1 to 10 carbon atoms), and halogen atoms, and each hydrogen atom of the cycloalkyl groups being optionally replaced by an alkyl group preferably having between 1 to 100 carbon atoms, e.g. 1 to 20 carbon atoms (e.g. 1 to 10 carbon atoms), or R¹ represents a polyethylene glycol (PEG) moiety having the formula C₂ₙR₄ₙ₊₂Oₙ₊₁ wherein n is an integer between 1 and 100; or R² and R³ form a ring together with carbon atoms to which R² and R³ are bonded;
R⁴ represents a hydrogen atom or a deuterium atom.

2. A compound according to claim 1, wherein R² and R³ each represent a phenyl group in the general formula (1A): 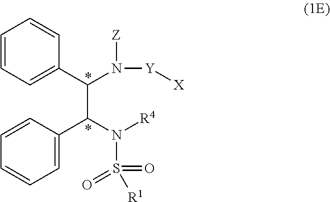
wherein each * represents an asymmetric carbon atom; and
X, Y, Z, R¹, and R⁴ are defined as above.

3. A compound according to claim 1, in which the asymmetric carbon atoms are represented by the general formula (1B): 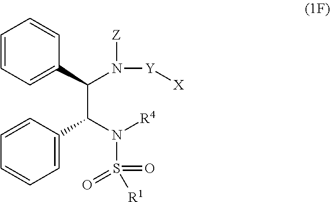
wherein X, Y, Z, R¹ and R⁴ are defined as above.

4. A compound according to claim 1, wherein R¹ represents one of p-tolyl group, a 2,4,6-trimethylphenyl group, a 4-trifluoromethylphenyl group, a pentafluorophenyl group, 4-methylbenzene, CH₃, CF₃, or 2,4,6-triisopropylbenzene.

5. A compound according to claim 1, wherein Y represents a CH₂ moiety and X represents a heterocyclic moiety (e.g. a five-membered heterocyclic ring) comprising one or more of O, S, Se, and/or P (e.g. a furan, a tetrahydrofuran, a thiophene, an isoxazole, a bromo-furan, or a thiazole).

6. A compound according to claim 1, wherein Y represents a CH₂ moiety and X represents a group selected from one of an ester (e.g. a t-butyl ester); a thioester; or an amide.

7. The compound of claim 1, wherein the compound is a metal-diamine complex, for example a transition metal-diamine complex, comprising a ligand according to claim 1 and represented by the general formula (2). 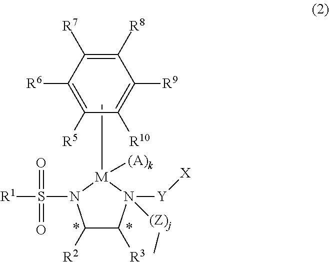
wherein
M represents a transition metal catalyst, and/or one of ruthenium, iridium, rhodium, or osmium;
each * represents an asymmetric carbon atom;
X, Y, Z, R¹, R², R³ are independently defined as in claim 1;
R⁵, R⁶, R⁷, R⁸, R⁹, and R¹⁰ each independently represent a group selected from a hydrogen atom, a deuterium atom, alkyl groups preferably having 1 to 100 carbon atoms, e.g. 1 to 10 carbon atoms, silyl groups preferably having 1 to 3 alkyl groups having 1 to 100 carbon atoms, e.g. 1 to 10 carbon atoms, alkoxy groups preferably having 1 to 100 carbon atoms, e.g. 1 to 10 carbon atoms, and C(═O)—OR¹¹, where R¹¹ represents an alkyl group preferably having 1 to 100 carbon atoms, e.g. 1 to 10 carbon atoms, a heteroaryl group preferably having 4 to 100 carbon atoms, e.g. 4 to 10 carbon atoms, or an aryl group preferably having 6 to 100 carbon atoms, e.g. 6 to 10 carbon atoms;
A represents a group selected from a trifluoromethanesulfonyloxy group, a p-toluenesulfonyloxy group, a methanesulfonyloxy group, a benzenesulfonyloxy group, a hydrogen atom, a halogen atom, acetate, hexafluorophosphate, and tetrafluoroborate;
j and k each represent 0 or 1, but j+k is not 1.

8. The compound of claim 1, wherein the compound is a metal-diamine complex, for example a transition metal-diamine complex comprising a ligand according to claim 1, and represented by the general formula (3): 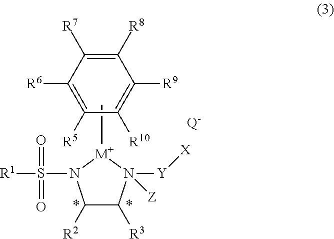
wherein
M represents a transition metal catalyst, and/or one of ruthenium, iridium, rhodium, or osmium;
each * represents an asymmetric carbon atom;
X, Y, Z, R¹, R², R³ are independently defined as in claim 1;
R⁵, R⁶, R⁷, R⁸, R⁹, and R¹⁰ each independently represent a group selected from a hydrogen atom, a deuterium atom, alkyl groups preferably having 1 to 100 carbon atoms, e.g. 1 to 10 carbon atoms, silyl groups preferably having 1 to 3 alkyl groups having 1 to 100 carbon atoms, e.g. 1 to 10 carbon atoms, alkoxy groups preferably having 1 to 100 carbon atoms, e.g. 1 to 10 carbon atoms, and C(═O)—OR¹¹, where R¹¹ represents an alkyl group preferably having 1 to 100 carbon atoms, e.g. 1 to 10 carbon atoms, a heteroaryl group preferably having 4 to 100 carbon atoms, e.g. 4 to 10 carbon atoms, or an aryl group preferably having 6 to 100 carbon atoms, e.g. 6 to 10 carbon atoms;
A represents a group selected from a trifluoromethanesulfonyloxy group, a p-toluenesulfonyloxy group, a methanesulfonyloxy group, a benzenesulfonyloxy group, a hydrogen atom, a halogen atom, acetate, hexafluorophosphate, and tetrafluoroborate;
Q represents a counter anion.

9. The compound of claim 1, wherein the compound is a metal-diamine complex, for example a transition metal-diamine complex, comprising a ligand according to claim 1 and represented by the general formula (4): 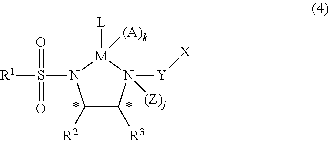
wherein
M represents a transition metal catalyst, and/or one of ruthenium, iridium, rhodium, or osmium;
each * represents an asymmetric carbon atom;
X, Y, Z, R¹, R², R³ are independently defined as in claim 1;
A represents a group selected from a trifluoromethanesulfonyloxy group, a p-toluenesulfonyloxy group, a methanesulfonyloxy group, a benzenesulfonyloxy group, a hydrogen atom, a halogen atom, acetate, hexafluorophosphate, and tetrafluoroborate;
j and k each represent 0 or 1, but j+k is not 1;
L represents a cyclopentadienyl or pentamethylcyclopentadienyl ligand.

10. The compound of claim 1, wherein the compound is a metal-diamine complex, for example a transition metal-diamine complex, comprising a ligand according to claim 1 and represented by the general formula (5): 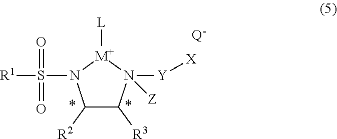
wherein
M represents a transition metal catalyst, and/or one of ruthenium, iridium, rhodium, or osmium;
each * represents an asymmetric carbon atom;
X, Y, Z, R¹, R², R³ are independently defined as in claim 1;
Q represents a counter anion;
L represents a cyclopentadienyl or pentamethylcyclopentadienyl ligand.

11. A method of producing a metal complex represented by one of the general formulae (2), (3), (4), or (5) according to claim 1, the method comprising reacting the ligand represented by the general formula (1), e.g. (1A) or (1B), with a metal compound, e.g. a ruthenium compound, an iridium compound, or a rhodium compound.

12. A method for selectively producing optically active compounds using one or more of the metal-diamine complexes (2), (3), (4), or (5) as a catalyst, the method comprising reducing a functional group of a substrate in the presence of one or more of complex (2), (3), (4), or (5) and a hydrogen source or hydrogen donor.

13. A method according to claim 12, wherein the hydrogen source is one or more of hydrogen gas, formic acid, isopropanol and/or a formate salt.

14. A method according to claim 12, wherein the functional group is an imine or an imino group, which is reduced to an amine.

15. A method according to claim 12, wherein the functional group is a ketone or a keto-group, which is reduced to an alcohol.
